SEQUENCE LISTING (25 sequences)

SEQ ID NO: 1 - DNA, 18 bp, Homo sapiens
  misc_feature: GSTP1_332_U18
tcggggattttagggcgt

SEQ ID NO: 2 - DNA, 21 bp, Homo sapiens
  misc_feature: GSTP1_513_L21
acgaaaactacgacgacgaaa

SEQ ID NO: 3 - DNA, 47 bp, Homo sapiens
  misc_feature: GSTP1 Scorpion
cgcacggcgaactcccgccgacgtgcgtgtagcggtcgtcggggttg

SEQ ID NO: 4 - DNA, 22 bp, Homo sapiens
  misc_feature: GSTPi Reverse Primer
gccccaatactaaatcacgacg

SEQ ID NO: 5 - DNA, 24 bp, Homo sapiens
  misc_feature: Actin_309_U24
gatataaggttagggataggatag

SEQ ID NO: 6 - DNA, 22 bp, Homo sapiens
  misc_feature: Actin_501_L22
aaccaataaaacctactcctcc

SEQ ID NO: 7 - DNA, 52 bp, Homo sapiens
  misc_feature: Actin Scorpion
ccgcgcatcaccaccccacacgcgcggggagtatataggttggggaagtttg

SEQ ID NO: 8 - DNA, 27 bp, Homo sapiens
  misc_feature: Actin Reverse Primer
aacacacaataacaaacacaaattcac

SEQ ID NO: 9 - DNA, 19 bp, Homo sapiens
  misc_feature: APC_Outer_692_U19
ccctataccccactacgaa

SEQ ID NO: 10 - DNA, 25 bp, Homo sapiens
  misc_feature: APC_Outer_830_L25
ggcgggttgtattaatatagttata

SEQ ID NO: 11 - DNA, 44 bp, Homo sapiens
  modified_base (1)..(1): texas red -
gccggcgggttttcgacgggccggccgaaccaaaacgctcccca

SEQ ID NO: 12 - DNA, 25 bp, Homo sapiens
  misc_feature: APC Lower Primer
gtcggttacgtgcgtttatatttag

SEQ ID NO: 13 - DNA, 25 bp, Homo sapiens
  misc_feature: RARB2_Outer_16_U25
ggaagtgagttgtttagaggtagga

SEQ ID NO: 14 - DNA, 25 bp, Homo sapiens
  misc_feature: RARB2_Outer_239_L25
tccaaacttactcgaccaatccaac

SEQ ID NO: 15 - DNA, 47 bp, Homo sapiens
  misc_feature: RARB2 Scorpion
cggcgcccgacgatacccaaagcgccgaacgcgagcgattcgagtag

SEQ ID NO: 16 - DNA, 24 bp, Homo sapiens
  misc_feature: RARB2 Lower Primer
cttacaaaaaaccttccgaatacg

SEQ ID NO: 17 - DNA, 4260 bp, Homo sapiens
  misc_feature: >gi|341173|gb|M24485.1|HUMGSTP1G Homo sapiens (clone pHGST-pi) glutathione S-transferase pi (GSTP1) gene, complete cds
aacaagagatcaatatctagaataaatggagatctgcaaatcaacagaaagtaggcagcaaagccaaagaaaatagcctaaggcacagccactaaaaggaacgtgatcatgtcctttgcagggacatgggtggagctggaagccgttagcctcagcaaactcacacaggaacagaaaaccagcgagaccgcatggtctcacttataagtgggagctgaacaatgagaacacatggtcacatggcggcgatcaacacacactggtgcctgttgagcggggtgctggggagggagagtaccaggaagaatagctaagggatactgggcttaatacctgggtgatgggatgatctgtacagcaaaccatcatggcgcacacacctatgtaacaaacctgcacatcctgcacatgtaccccagaacttcaaataaaagttggacggccaggcgtggtggctcacgcctgtaatcccagcactttgggaagccgaggcgtgcagatcacctaaggtcaggagttcgagaccagcccggccaacatggtgaaaccccgtctctactaaaaatacaaaaatcagccagatgtggcacgcacctataattccacctactcgggaggctgaagcagaattgcttgaacccgagaggcggaggttgcagtgagccgccgagatcgcgccactgcactccagcctgggccacagcgtgagactacgtcataaaataaaataaaataacacaaaataaaataaaataaaataaaataaaataaaataataaaataaaataaaataaaataaaataaaataaaataaagcaatttcctttcctctaagcggcctccacccctctcccctgccctgtgaagcgggtgtgcaagctccgggatcgcagcggtcttagggaatttccccccgcgatgtcccggcgcgccagttcgctgcgcacacttcgctgcggtcctcttcctgctgtctgtttactccctaggccccgctggggacctgggaaagagggaaaggcttccccggccagctgcgcggcgactccggggactccagggcgcccctctgcggccgacgcccggggtgcagcggccgccggggctggggccggcgggagtccgcgggaccctccagaagagcggccggcgccgtgactcagcactggggcggagcggggcgggaccacccttataaggctcggaggccgcgaggccttcgctggagtttcgccgccgcagtcttcgccaccagtgagtacgcgcggcccgctccccggggatggggctcagagctcccagcatggggccaacccgcagcatcaggcccgggctcccggcagggctcctcgcccacctcgagacccgggacgggggcctaggggacccaggacgtccccagtgccgttagcggctttcagggggcccggagcgcctcggggagggatgggaccccgggggcggggagggggggcaggctgcgctcaccgcgccttggcatcctcccccgggctccagcaaacttttctttgttcgctgcagtgccgccctacaccgtggtctatttcccagttcgaggtaggagcatgtgtctggcagggaagggaggcaggggctggggctgcagcccacagcccctcgcccacccggagagatccgaacccccttatccctccgtcgtgtggcttttaccccgggcctccttcctgttccccgcctctcccgccatgcctgctccccgccccagtgttgtgtgaaatcttcggaggaacctgtttacctgttccctccctgcactcctgacccctccccgggttgctgcgaggcggagtcggcccggtccccacatctcgtacttctccctccccgcaggccgctgcgcggccctgcgcatgctgctggcagatcagggccagagctggaaggaggaggtggtgaccgtggagacgtggcaggagggctcactcaaagcctcctgcgtaagtgaccatgcccgggcaaggggagggggtgctgggccttagggggctgtgactaggatcgggggacgcccaagctcagtgcccctccctgagccatgcctcccccaacagctatacgggcagctccccaagttccaggacggagacctcaccctgtaccagtccaataccatcctgcgtcacctgggccgcacccttggtgagtcttgaacctccaagtccagggcaggcatgggcaagcctctgcccccggagcccttttgtttaaatcagctgccccgcagccctctggagtggaggaaactgagacccactgaggttacgtagtttgcccaaggtcaagcctgggtgcctgcaatccttgccctgtgccaggctgcctcccaggtgtcaggtgagctctgagcacctgctgtgtggcagtctctcatccttccacgcacatcctcttcccctcctcccaggctggggctcacagacagccccctggttggcccatccccagtgactgtgtgttgatcaggcgcccagtcacgcggcctgctcccctccacccaaccccagggctctatgggaaggaccagcaggaggcagccctggtggacatggtgaatgacggcgtggaggacctccgctgcaaatacatctccctcatctacaccaactatgtgagcatctgcaccagggttgggcactgggggctgaacaaagaaaggggcttcttgtgccctcaccccccttacccctcaggtggcttgggctgaccccttcttgggtcagggtgcaggggctgggtcagctctgggccaggggcccaggggcctgggacaagacacaacctgcacccttattgcctgggacatcaaccagccaagtaacgggtcatgggggcgagtgcaaggacagagacctccagcaactggtggtttctgatctcctggggtggcgagggcttcctggagtagccagaggtggaggaggatttgtcgccagtttctggatggaggtgctggcacttttagctgaggaaaatatgcagacacagagcacatttggggacctgggaccagttcagcagaggcagcgtgtgtgcgcgtgcgtgtgcgtgtgtgtgcgtgtgtgtgtgtacgcttgcatttgtgtcgggtgggtaaggagatagagatgggcgggcagtaggcccaggtcccgaaggccttgaacccactggtttggagtctcctaagggcaatgggggccattgagaagtctgaacagggctgtgtctgaatgtgaggtctagaaggatcctccagagaagccagctctaaagcttttgcaatcatctggtgagagaacccagcaaggatggacaggcagaatggaatagagatgagttggcagctgaagtggacaggatttggtactagcctggttgtggggagcaagcagaggagaatctgggactctggtgtctggcctggggcagacgggggtgtctcaggggctgggagggatgagagtaggatgatacatggtggtgtctggcaggaggcgggcaaggatgactatgtgaaggcactgcccgggcaactgaagccttttgagaccctgctgtcccagaaccagggaggcaagaccttcattgtgggagaccaggtgagcatctggccccatgctgttccttcctcgccaccctctgcttccagatggacacaggtgtgagccatttgtttagcaaagcagagcagacctaggggatgggcttaggccctctgcccccaattcctccagcctgctcccgctggctgagtccctagcccccctgccctgcagatctccttcgctgactacaacctgctggacttgctgctgatccatgaggtcctagcccctggctgcctggatgcgttccccctgctctcagcatatgtggggcgcctcagtgcccggcccaagctcaaggccttcctggcctcccctgagtacgtgaacctccccatcaatggcaacgggaaacagtgagggttggggggactctgagcgggaggcagagtttgccttcctttctccaggaccaataaaatttctaagagagctactatgagcactgtgtttcctgggacggggcttaggggttctcagcctcgaggtcggtgggagggcagagcagaggactagaaaacagctcctccagcacagtcagtggcttcctggagccctcagcctggctgtgtttactgaacctcacaaactagaagaggaagaaaaaaaaagagagagagaaacaaagagaaat

SEQ ID NO: 18 - DNA, 866 bp, Homo sapiens
  misc_feature: APC
acttatatatctgacagttgatttgtcctcacctctaaattggaatttaagcatcacctggttcgatttaatgcaatgtagaatttgcattaaaatactacattaaagcctcagatttgtagtagctaacagcacttctatgtatgtgtcagggactgctctaaatacttcatatatattaactcctctattctgtacttctgttcccgttttatacagcaggaaattgaaacactgagaggttaagtaactaaagttacagagctagagtgacaggagtaaagcttcaactcaggcaacccagacgtccagagntctgatctccactactaagctgctagcatagcttttctggtaactatttttaattcaatataattcgaatgatctatctaacaagtcatcactctgacaactcagtgacttgtaatgtaaaattattcattgtaattcacttaatattattgtttctctgtgctgcaaaaatcatagcaatcgagatgtaatttattactctccctcccacctccggcatcttgtgctaatccttctgccctgcggacctcccccgactctttactatgcgtgtcaactgccatcaacttccttgcttgctggggactggggccgtgagggcatacccccgaggggtacggggctagggctaggcaggctgtgcggttgggcggggccctgtgccccactgcggagtgcgggtcgggaagcggagagagaagcagctgtgtaatccgctggatgcggaccagggcgctccccattcccgtcgggagcccgccgattggctgggtgtgggcgcacgtgaccgacatgtggctgtattggtgcagcccgccagggtgtcactg

SEQ ID NO: 19 - DNA, 10386 bp, Homo sapiens
  misc_feature: >gi|21626462|ref|NM_000038.2| Homo sapiens adenomatosis polyposis coli (APC), mRNA
attgaggactcggaaatgaggtccaagggtagccaaggatggctgcagcttcatatgatcagttgttaaagcaagttgaggcactgaagatggagaactcaaatcttcgacaagagctagaagataattccaatcatcttacaaaactggaaactgaggcatctaatatgaaggaagtacttaaacaactacaaggaagtattgaagatgaagctatggcttcttctggacagattgatttattagagcgtcttaaagagcttaacttagatagcagtaatttccctggagtaaaactgcggtcaaaaatgtccctccgttcttatggaagccgggaaggatctgtatcaagccgttctggagagtgcagtcctgttcctatgggttcatttccaagaagagggtttgtaaatggaagcagagaaagtactggatatttagaagaacttgagaaagagaggtcattgcttcttgctgatcttgacaaagaagaaaaggaaaaagactggtattacgctcaacttcagaatctcactaaaagaatagatagtcttcctttaactgaaaatttttccttacaaacagatatgaccagaaggcaattggaatatgaagcaaggcaaatcagagttgcgatggaagaacaactaggtacctgccaggatatggaaaaacgagcacagcgaagaatagccagaattcagcaaatcgaaaaggacatacttcgtatacgacagcttttacagtcccaagcaacagaagcagagaggtcatctcagaacaagcatgaaaccggctcacatgatgctgagcggcagaatgaaggtcaaggagtgggagaaatcaacatggcaacttctggtaatggtcagggttcaactacacgaatggaccatgaaacagccagtgttttgagttctagtagcacacactctgcacctcgaaggctgacaagtcatctgggaaccaaggtggaaatggtgtattcattgttgtcaatgcttggtactcatgataaggatgatatgtcgcgaactttgctagctatgtctagctcccaagacagctgtatatccatgcgacagtctggatgtcttcctctcctcatccagcttttacatggcaatgacaaagactctgtattgttgggaaattcccggggcagtaaagaggctcgggccagggccagtgcagcactccacaacatcattcactcacagcctgatgacaagagaggcaggcgtgaaatccgagtccttcatcttttggaacagatacgcgcttactgtgaaacctgttgggagtggcaggaagctcatgaaccaggcatggaccaggacaaaaatccaatgccagctcctgttgaacatcagatctgtcctgctgtgtgtgttctaatgaaactttcatttgatgaagagcatagacatgcaatgaatgaactagggggactacaggccattgcagaattattgcaagtggactgtgaaatgtacgggcttactaatgaccactacagtattacactaagacgatatgctggaatggctttgacaaacttgacttttggagatgtagccaacaaggctacgctatgctctatgaaaggctgcatgagagcacttgtggcccaactaaaatctgaaagtgaagacttacagcaggttattgcaagtgttttgaggaatttgtcttggcgagcagatgtaaatagtaaaaagacgttgcgagaagttggaagtgtgaaagcattgatggaatgtgctttagaagttaaaaaggaatcaaccctcaaaagcgtattgagtgccttatggaatttgtcagcacattgcactgagaataaagctgatatatgtgctgtagatggtgcacttgcatttttggttggcactcttacttaccggagccagacaaacactttagccattattgaaagtggaggtgggatattacggaatgtgtccagcttgatagctacaaatgaggaccacaggcaaatcctaagagagaacaactgtctacaaactttattacaacacttaaaatctcatagtttgacaatagtcagtaatgcatgtggaactttgtggaatctctcagcaagaaatcctaaagaccaggaagcattatgggacatgggggcagttagcatgctcaagaacctcattcattcaaagcacaaaatgattgctatgggaagtgctgcagctttaaggaatctcatggcaaataggcctgcgaagtacaaggatgccaatattatgtctcctggctcaagcttgccatctcttcatgttaggaaacaaaaagccctagaagcagaattagatgctcagcacttatcagaaacttttgacaatatagacaatttaagtcccaaggcatctcatcgtagtaagcagagacacaagcaaagtctctatggtgattatgtttttgacaccaatcgacatgatgataataggtcagacaattttaatactggcaacatgactgtcctttcaccatatttgaatactacagtgttacccagctcctcttcatcaagaggaagcttagatagttctcgttctgaaaaagatagaagtttggagagagaacgcggaattggtctaggcaactaccatccagcaacagaaaatccaggaacttcttcaaagcgaggtttgcagatctccaccactgcagcccagattgccaaagtcatggaagaagtgtcagccattcatacctctcaggaagacagaagttctgggtctaccactgaattacattgtgtgacagatgagagaaatgcacttagaagaagctctgctgcccatacacattcaaacacttacaatttcactaagtcggaaaattcaaataggacatgttctatgccttatgccaaattagaatacaagagatcttcaaatgatagtttaaatagtgtcagtagtagtgatggttatggtaaaagaggtcaaatgaaaccctcgattgaatcctattctgaagatgatgaaagtaagttttgcagttatggtcaatacccagccgacctagcccataaaatacatagtgcaaatcatatggatgataatgatggagaactagatacaccaataaattatagtcttaaatattcagatgagcagttgaactctggaaggcaaagtccttcacagaatgaaagatgggcaagacccaaacacataatagaagatgaaataaaacaaagtgagcaaagacaatcaaggaatcaaagtacaacttatcctgtttatactgagagcactgatgataaacacctcaagttccaaccacattttggacagcaggaatgtgtttctccatacaggtcacggggagccaatggttcagaaacaaatcgagtgggttctaatcatggaattaatcaaaatgtaagccagtctttgtgtcaagaagatgactatgaagatgataagcctaccaattatagtgaacgttactctgaagaagaacagcatgaagaagaagagagaccaacaaattatagcataaaatataatgaagagaaacgtcatgtggatcagcctattgattatagtttaaaatatgccacagatattccttcatcacagaaacagtcattttcattctcaaagagttcatctggacaaagcagtaaaaccgaacatatgtcttcaagcagtgagaatacgtccacaccttcatctaatgccaagaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtgttgaagggacacctataaacttttccacagctacatctctaagtgatctaacaatcgaatcccctccaaatgagttagctgctggagaaggagttagaggaggagcacagtcaggtgaatttgaaaaacgagataccattcctacagaaggcagaagtacagatgaggctcaaggaggaaaaacctcatctgtaaccatacctgaattggatgacaataaagcagaggaaggtgatattcttgcagaatgcattaattctgctatgcccaaagggaaaagtcacaagcctttccgtgtgaaaaagataatggaccaggtccagcaagcatctgcgtcgtcttctgcacccaacaaaaatcagttagatggtaagaaaaagaaaccaacttcaccagtaaaacctataccacaaaatactgaatataggacacgtgtaagaaaaaatgcagactcaaaaaataatttaaatgctgagagagttttctcagacaacaaagattcaaagaaacagaatttgaaaaataattccaaggacttcaatgataagctcccaaataatgaagatagagtcagaggaagttttgcttttgattcacctcatcattacacgcctattgaaggaactccttactgtttttcacgaaatgattctttgagttctctagattttgatgatgatgatgttgacctttccagggaaaaggctgaattaagaaaggcaaaagaaaataaggaatcagaggctaaagttaccagccacacagaactaacctccaaccaacaatcagctaataagacacaagctattgcaaagcagccaataaatcgaggtcagcctaaacccatacttcagaaacaatccacttttccccagtcatccaaagacataccagacagaggggcagcaactgatgaaaagttacagaattttgctattgaaaatactccagtttgcttttctcataattcctctctgagttctctcagtgacattgaccaagaaaacaacaataaagaaaatgaacctatcaaagagactgagccccctgactcacagggagaaccaagtaaacctcaagcatcaggctatgctcctaaatcatttcatgttgaagataccccagtttgtttctcaagaaacagttctctcagttctcttagtattgactctgaagatgacctgttgcaggaatgtataagctccgcaatgccaaaaaagaaaaagccttcaagactcaagggtgataatgaaaaacatagtcccagaaatatgggtggcatattaggtgaagatctgacacttgatttgaaagatatacagagaccagattcagaacatggtctatcccctgattcagaaaattttgattggaaagctattcaggaaggtgcaaattccatagtaagtagtttacatcaagctgctgctgctgcatgtttatctagacaagcttcgtctgattcagattccatcctttccctgaaatcaggaatctctctgggatcaccatttcatcttacacctgatcaagaagaaaaaccctttacaagtaataaaggcccacgaattctaaaaccaggggagaaaagtacattggaaactaaaaagatagaatctgaaagtaaaggaatcaaaggaggaaaaaaagtttataaaagtttgattactggaaaagttcgatctaattcagaaatttcaggccaaatgaaacagccccttcaagcaaacatgccttcaatctctcgaggcaggacaatgattcatattccaggagttcgaaatagctcctcaagtacaagtcctgtttctaaaaaaggcccaccccttaagactccagcctccaaaagccctagtgaaggtcaaacagccaccacttctcctagaggagccaagccatctgtgaaatcagaattaagccctgttgccaggcagacatcccaaataggtgggtcaagtaaagcaccttctagatcaggatctagagattcgaccccttcaagacctgcccagcaaccattaagtagacctatacagtctcctggccgaaactcaatttcccctggtagaaatggaataagtcctcctaacaaattatctcaacttccaaggacatcatcccctagtactgcttcaactaagtcctcaggttctggaaaaatgtcatatacatctccaggtagacagatgagccaacagaaccttaccaaacaaacaggtttatccaagaatgccagtagtattccaagaagtgagtctgcctccaaaggactaaatcagatgaataatggtaatggagccaataaaaaggtagaactttctagaatgtcttcaactaaatcaagtggaagtgaatctgatagatcagaaagacctgtattagtacgccagtcaactttcatcaaagaagctccaagcccaaccttaagaagaaaattggaggaatctgcttcatttgaatctctttctccatcatctagaccagcttctcccactaggtcccaggcacaaactccagttttaagtccttcccttcctgatatgtctctatccacacattcgtctgttcaggctggtggatggcgaaaactcccacctaatctcagtcccactatagagtataatgatggaagaccagcaaagcgccatgatattgcacggtctcattctgaaagtccttctagacttccaatcaataggtcaggaacctggaaacgtgagcacagcaaacattcatcatcccttcctcgagtaagcacttggagaagaactggaagttcatcttcaattctttctgcttcatcagaatccagtgaaaaagcaaaaagtgaggatgaaaaacatgtgaactctatttcaggaaccaaacaaagtaaagaaaaccaagtatccgcaaaaggaacatggagaaaaataaaagaaaatgaattttctcccacaaatagtacttctcagaccgtttcctcaggtgctacaaatggtgctgaatcaaagactctaatttatcaaatggcacctgctgtttctaaaacagaggatgtttgggtgagaattgaggactgtcccattaacaatcctagatctggaagatctcccacaggtaatactcccccggtgattgacagtgtttcagaaaaggcaaatccaaacattaaagattcaaaagataatcaggcaaaacaaaatgtgggtaatggcagtgttcccatgcgtaccgtgggtttggaaaatcgcctgaactcctttattcaggtggatgcccctgaccaaaaaggaactgagataaaaccaggacaaaataatcctgtccctgtatcagagactaatgaaagttctatagtggaacgtaccccattcagttctagcagctcaagcaaacacagttcacctagtgggactgttgctgccagagtgactccttttaattacaacccaagccctaggaaaagcagcgcagatagcacttcagctcggccatctcagatcccaactccagtgaataacaacacaaagaagcgagattccaaaactgacagcacagaatccagtggaacccaaagtcctaagcgccattctgggtcttaccttgtgacatctgtttaaaagagaggaagaatgaaactaagaaaattctatgttaattacaactgctatatagacattttgtttcaaatgaaactttaaaagactgaaaaattttgtaaataggtttgattcttgttagagggtttttgttctggaagccatatttgatagtatactttgtcttcactggtcttattttgggaggcactcttgatggttaggaaaaaaatagtaaagccaagtatgtttgtacagtatgttttacatgtatttaaagtagcatcccatcccaacttcctttaattattgcttgtcttaaaataatgaacactacagatagaaaatatgatatattgctgttatcaatcatttctagattataaactgactaaacttacatcagggaaaaattggtatttatgcaaaaaaaaatgtttttgtccttgtgagtccatctaacatcataattaatcatgtggctgtgaaattcacagtaatatggttcccgatgaacaagctttacccagcctgtttgctttactgcatgaatgaaactgatggttcaatttcagaagtaatgattaacagttatgtggtcacatgatgtgcatagagatagctacagtgtaataatttacactattttgtgctccaaacaaaacaaaaatctgtgtaactgtaaaacattgaatgaaactattttacctgaactagattttatctgaaagtaggtagaatttttgctatgctgtaatttgttgtatattctggtatttgaggtgagatggctgctcttttattaatgagacatgaattgtgtctcaacagaaactaaatgaacatttcagaataaattattgctgtatgtaaactgttactgaaattggtatttgtttgaagggtcttgtttcacatttgtattaataattgtttaaaatgcctcttttaaaagcttatataaattttttncttcagcttctatgcattaagagtaaaattcctcttactgtaataaaaacaattgaagaagactgttgccacttaaccattccatgcgttggcacttatctattcctgaaattcttttatgtgattagctcatcttgatttttaacatttttccacttaaacttttttttcttactccactggagctcagtaaaagtaaattcatgtaatagcaatgcaagcagcctagcacagactaagcattgagcataataggcccacataatttcctctttcttaatattatagaaattctgtacttgaaattgattcttagacattgcagtctcttcgaggctttacagtgtaaactgtcttgccccttcatcttcttgttgcaactgggtctgacatgaacactttttatcaccctgtatgttagggcaagatctcagcagtgaagtataatcagcactttgccatgctcagaaaattcaaatcacatggaactttagaggtagatttaatacgattaagatattcagaagtatattttagaatccctgcctgttaaggaaactttatttgtggtaggtacagttctggggtacatgttaagtgtccccttatacagtggagggaagtcttccttcctgaaggaaaataaactgacacttattaactaagataatttacttaatatatcttccctgatttgttttaaaagatcagagggtgactgatgatacatgcatacatatttgttgaataaatgaaaatttatttttagtgataagattcatacactctgtatttggggagagaaaacctttttaagcatggtggggcactcagataggagtgaatacacctacctggtggtcat

SEQ ID NO: 20 - DNA, 2762 bp, Homo sapiens
  misc_feature: RARB2       >gi|14916495|ref|NM_016152.2| Homo sapiens retinoic acid receptor, beta (RARB), transcript variant 2, mRNA
gtgacagaagtagtaggaagtgagctgttcagaggcaggagggtctattctttgccaaaggggggaccagaattccccatgcgagctgtttgaggactgggatgccgagaacgcgagcgatccgagcagggtttgtctgggcaccgtcggggtaggatccggaacgcattcggaaggctttttgcaagcatttacttggaaggagaacttgggatctttctgggaaccccccgccccggctggattggccgagcaagcctggaaaatgcaattgaaacacagagcaccagctctgaggaactcgtcccaagccccccatctccacttcctccccctcgagtgtacaaaccctgcttcgtctgccaggacaaatcatcagggtaccactatggggtcagcgcctgtgagggatgtaagggctttttccgcagaagtattcagaagaatatgatttacacttgtcaccgagataagaactgtgttattaataaagtcaccaggaatcgatgccaatactgtcgactccagaagtgctttgaagtgggaatgtccaaagaatctgtcaggaatgacaggaacaagaaaaagaaggagacttcgaagcaagaatgcacagagagctatgaaatgacagctgagttggacgatctcacagagaagatccgaaaagctcaccaggaaactttcccttcactctgccagctgggtaaatacaccacgaattccagtgctgaccatcgagtccgactggacctgggcctctgggacaaattcagtgaactggccaccaagtgcattattaagatcgtggagtttgctaaacgtctgcctggtttcactggcttgaccatcgcagaccaaattaccctgctgaaggccgcctgcctggacatcctgattcttagaatttgcaccaggtataccccagaacaagacaccatgactttctcagacggccttaccctaaatcgaactcagatgcacaatgctggatttggtcctctgactgaccttgtgttcacctttgccaaccagctcctgcctttggaaatggatgacacagaaacaggccttctcagtgccatctgcttaatctgtggagaccgccaggaccttgaggaaccgacaaaagtagataagctacaagaaccattgctggaagcactaaaaatttatatcagaaaaagacgacccagcaagcctcacatgtttccaaagatcttaatgaaaatcacagatctccgtagcatcagtgctaaaggtgcagagcgtgtaattaccttgaaaatggaaattcctggatcaatgccacctctcattcaagaaatgctggagaattctgaaggacatgaacccttgaccccaagttcaagtgggaacacagcagagcacagtcctagcatctcacccagctcagtggaaaacagtggggtcagtcagtcaccactcgtgcaataagacattttctagctacttcaaacattccccagtaccttcagttccaggatttaaaatgcaagaaaaaacatttttactgctgcttagtttttggactgaaaagatattaaaactcaagaaggaccaagaagttttcatatgtatcaatatatatactcctcactgtgtaacttacctagaaatacaaacttttccaattttaaaaaatcagccatttcatgcaaccagaaactagttaaaagcttctattttcctctttgaacactcaagatgcatggcaaagacccagtcaaaatgatttacccctggttaagtttctgaagactttgtacatacagaagtatggctctgttctttctatactgtatgtttggtgctttccttttgtcttgcatactcaaaataaccatgacaccaaggttatgaaatagactactgtacacgtctacctaggttcaaaaagataactgtcttgctttcatggaatagtcaagacatcaaggtaaggaaacaggactattgacaggactattgtacagtatgacaagataaggctgaagatattctactttagttagtatggaagcttgtctttgctctttctgatgctctcaaactgcatcttttatttcatgttgcccagtaaaagtatacaaattccctgcactagcagaagagaattctgtatcagtgtaactgccagttcagttaatcaaatgtcatttgttcaattgttaatgtcactttaaattaaaagtggtttattacttgtttaatgacataactacacagttagttaaaaaaaatttttttacagtaatgatagcctccaaggcagaaacacttttcagtgttaagtttttgtttacttgttcacaagccattagggaaatttcatgggataattagcaggctggtctaccactggaccatgtaactctagtgtccttcctgattcatgcctgatattgggatttttttccagcccttcttgatgccaagggctaattatattacatcccaaagaaacaggcatagaatctgcctcctttgaccttgttcaatcactatgaagcagagtgaaagctgtggtagagtggttaacagatacaagtgtcagtttcttagttctcatttaagcactactggaatttttttttttgatatattagcaagtctgtgatgtactttcactggctctgtttgtacattgagattgtttgtttaacaatgctttctatgttcatatactgtttacctttttccatggactctcctggcaaagaataaaatatatttatttt

SEQ ID NO: 21 - DNA, 8670 bp, Homo sapiens
  misc_feature: S100A2 gene
gagctcaagagttcaagacccgtctgggcaagatggcaaaactccatcaccacaaaagatgcaaaaagatgcgcacagtggcgcacacctatagccccagttactgaggaggttaatgtgggaggatcacatgaggctgcagtgagctgtgatggtgccactgtactccagccttggcgacagtgagtctatgtctcaaataagtaagtaaacaaaaattaaaaagaatccagtccacagggcatttgaaggcaagaggaaaagatgccagaatcagagatggggagaagatgggcttcacgcacctgctgaggttgagaaatgagacagataggctgagtgtggggtggagagaggatgggcagagagactgaggctggtctgaatggaaatgaaatgttagggctctcagggttatcggggaataattggagcttctaggaaaggtttaacgttgtgaccacctgtgtgcgtcatgcctccccaccccttactaattgtgtgaatttggcagactttgagtctcagtgttctcctctgtgaagtggggtcatcttattccaactcctgggattgttgtgtgaattaaatggggtaatgtacggagagcacctgacgcacagcgagtgcttcaaaatttcagtctgcaccccccagcaaaggatatgcacacgcccattgtgagtgacaaatccaggatgacctgaacccaatgtgataacgtgggtcctcgcatgctggtcatgctgccgggagacacttatggatccaattagtacaacaggggaaataaattatttaatgcattttgctaagacagaatacctcagaacttattttgtggggtggggcataataaagggggtccttctgctgaaaacgtttaagctcaggttcgtggcaccactcaaccaaggtcgacagtcacacagtaagccagaggcaatgtcaggacttaaactaaacctgtggcccccacaatgaggccatttctctttcccctgaacggcctggggaaagggggtgggtgggcagaacttggcagtggccaatccctcacttctgtcccctggttttctcctgcccttatctctaggcttgcattgattgattgattgagacagggtcttgctctgtcgtccaggctggagtgcagtggcacgatcatggctcactgcagcctcaaactcctaggctcaagtggtctttccgcctcctatctcccgagtacccatatccctaggcttttaaaatggcttccaggtatctggctgccgtctcagacatccacctgggcttctgggcagggactgtccgggaaacctcatctatgtgaagcaggtgtgggtgtaggaaggccgcttggaaatgaatcagcactgtctcctgtttgagtcgtaagcagggcgccagagggtctggcggacaagaaagggaggatgacaggaggccggcactgcaatgacacgccttagccaccagagggcacgaagcagctgggcaaaatcccgcggggcccctggtggaaaatttctggcacctggagcccggagatggggtggacggaatgtgaggacccagcttcctgaggctgggccggggcagagtcactgctttggatgtccgcagggcctgcttgtgtcttgactactctgcctttgtagacagctggagaatgtgagagtgggattgggatcggactctagggccattccgtacaactctcctgccctgccgtgggggagggagttgcccaaggttacgcagcaagttagtggcaaatgaatacgattatcaccagtctcaggtatatggccatttgatgggcgcagtcgcagcctcagttcctgagacagagacacctgattaaggacaggccttcaggagctgaccctagtgacccgcggctctgctgctgtctctgtttttctccctggcttttccatctgactgactctttgtcttcttcgtctgcctgcctgtctccgtctctgcccgctggggggtttgctcaactccctcactgggtcctgggagccgcagtttcctgctgtcactcctcagggatttgtagctctctgaagctcttttccgacccgttgtctcggttccactcttgggatccagaggagaggtgattatttcgtagcatagtcagtggtgtgatttcacggggtgagaaggactcccttgctcctaagcactcctccagtgacccctgttgccatgtggtagccgtaagcactggttggcacctggtgtgggcgagacccttacctcatgcagaaatgagtaagactggtgagctcactatgtggggtgaggctgagagaaaacaagtacacaggtgattcagtcaaaatcagaattctctaagtacacacgaaaagggcaaaaggggcgctttgtacaggacagaacaggtagacactgaatccggttgggccctgggaaggctccctgcagtggcctttgaagggggggttggatttcagcaggatagagggcatgggcatgtgtgggcacgttctgaacagaggggtcagcgcaagccgagggtcttggccacactagttgcatgtgccggtgtgtttaagggacacgcagcagcaggccgagtctggagcgcctcactgccaggctttttaaaaatttttaattttaatttaattttattttatttttactttaagttctggcatacatgtgcagaatgtggtttgttacataggtatacatgtgccatggtggtttgctgcacctatcaacccatcatctaggttttaagccccgcatgcatcaggtattagtcctaatgctctccctccccttgcccccatccttctccccgcaactgcccacaggccctggtatgtggtgttcccttccctgtgtccatatgttctcattgttcaactcccacttatgagtgagaacataccgcctggctttaagggacagccatggggatgcactgcagtttctgagcagggaaggccctgtggaggcccttagttaaaaggaaagaatggctgtgaaaatcgatgcattgcgctcccttgtccctcaccctcagtgtgaagggtttttattccgagttctacttgaagtaggcctcgatgggaagacaagtagcatgaggggttcaagtactgaggggagcaagggacactcggtggctgtgccaaggtgtagaagaggacactgggggccccaagacctgacttcatgtacactgctcaggctggcccccaagtcacacggtgaccgctaggaagggaccagcctgttctcagtctgatcctacagccatgtcattatccaaagctcctcctggcagggcctgtttggggtctctgtgccagtgctttccctgccaggctgggctggggcttccacctactgctctgggactgctgctgccctggccctgggggaggagggtgtgccgctgagtcactgcctgggcatctgggcctggaacctcgggtgagtcacttagggctgaggtagaggggctgggggaggggaagaagctactcgacagctggagcagggaggggagctggggccacaggaagggcggtgccctgatgcccagacgggccgggatagacaaagggccaaggaggaaggggccctgggagggggcagccctcccttgggctggggtctgaatggcacagtgtttgcctttctccgggtctggggaggacatgtgtgtggggggcagtgagagagggctgtggctgagggctgtgcttcaggcctggattctggcttgggaagctgtccagctggtgttttcagccttgggtagggatgtacccctacccacccacccagccctcaagctggagaagaggaggccaaagttttcctgttcagcctttaactactcgggacttccttatgctccccacagactgtggcccagcccaactgcggctgtgtgtagagcaaccccatttctcactgcttccccatccttccagacaccttcctacacagagggaccttcccaggtatttctaagcacacttagttacctcattacctcattaagaggtattctggtgctggccattaaaagtcactccacttcatccatgccctgaagtcagtcctgtccttctcctcctgatgtcccccagctgcctcctctggcccccagcttcctaaggtggccccaggttgcttctctctcacacacacgggcgcatgtatgtacacgagcactggaccatgaagtctcagcgtgtgctcacagcctctcacacaggagtgggctgtgactcacaggcatgtcatgagaatgaggcctggcaccagtctccaggccccagagcaggggttgcctcccctcaccccggtccaggatgcccagtccccacgacacctcccacttcccactgtggcctgggtgggctcaggggctgcccttgacctggcctagagccctcccccagctggtggtggagctggcactctctgggagggagggggctgggagggaatgagtgggaatggcaagaggccagggtttggtgggatcaggttgaggcaggtttggtttccttaaaatgccaagttgggggccagtggggcccacatataaatcctcaccctgggagcctggctgccttgctctccttcctgggtctgtctctgccacctggtctggtgagtacctctgtcctgctgagggcagggtggggaggatccccgtgggtctctgtctttgtctccacagttctctcattccagcttccctggtgggatcaacctgggcctctctgggccttcccccttggaagaactctctgtgaagtgctgaagtgttgactgaagggttttttttttttttttttttttttgagatggagtctcgctctgtcgcccaggctggagtacagtggtgtgatctcagctcactgcaaactccccctcccaggttcacgccatttccctgcctcagcctcccgagtagctgggactgcaggcgcccaccaccatgcccggctaatttttttgtatttttagtagagatggggtttcaccatgttagccaggatggtctcgatctcctgatctcgtgatccacccatctcggcctcccaaagtgctgggattacaggagtaagccaccgcgcccggccgactgaagggtttttctccaggttcctctgtgaggtctcagtgcaggggttgctctgaggccctcccctggatatctcagtctaggggcccttctttgggggtctaggcctaggagcaggaggtgtgcatgtgggcgttgctgcaaaaagaatcctgagatttttttttttttttttttttttgcaaagtcctggattctagcaggactaaggtgcaagaggcaggggtctcaagactctgcctgggtcatggccccaagcagcaaagctctgccccctgcctcggtgaaggcagggctggcatgatgggcccagggcatgccctgcctctggcatagctcctctggcctcaccctgaaacctgcctaacctttccaggctggtctgagtattctcagaggccttgccgctgaggtctgtcccatcctgatcccaaggcaatgaacatttcatatctttaattctaattccaacaggatccttcctggtggagagaatgttaagttgcccccaccctatccatgcccctgtctgcctagaggctcaggggccttcagggtgaggggagacacattccccaccctctgggagctcctagtctgagagaggaaacactcctgcccaagggagcttccagttagatggcagagagagatgcctctggcttcaggagtcccgagtctaaggagggaaacgactccttcagggagcttcctgctcctaggctgtagccatggctcctgccagactgcacaggagcccccatctgccagccggtgcatgtggccctgctccccagagcctgcgcagatgccatcaaaatgggactctggtcaccctgtcatttcccttctggcagacactaaaatggggagccctgccctcaggggggtgtcccaagtgccatcagaggaggcttggtgactcccagacacaagggaagctttagcgtctgccctcagggtgagatggaggtatccctccggcctcagggaaccacagtctgaggggagatgcagcccctgccttcccattcagagaggggttttgtgaggtggcttgggggcatagggcagaagtggatcctacaggctgagctaaggccccaagagcctcagcagtgtacccatcacctggcacctctgcagccacagatccatgatgtgcagttctctggagcaggcgctggctgtgctggtcactaccttccacaagtactcctgccaagagggcgacaagttcaagctgagtaagggggaaatgaaggaacttctgcacaaggagctgcccagctttgtgggggtgagtggcacaggcctgtgggggaggtcctggtgtgagtgtgggggtgcaggttaaatctctcccccagttccgggtgcctgtcgatgcaggtgccagggtggggcccagcccctccccactttagcttcatggctccactggagtggaaatgaggcccgagtgggagtgcttaattaatggctgtttcctgcaacattccagagaaccatgtgctgtgagggccttccgagtccatctgtttaatcctgtcattggaacttgagaaaccagagcccagaagggaaaagtgattgtcccaagatcacacagcactggcacgttctctctctctcttttcttttctttttttttttttgagatggagtttccctcttgttgcccaggctggagtgcaatggcacgatctcggctcactgcaacctctgcctccaggggtcaagcaattctcctgtctcagcctcctgagtagctgggactacaggcgcatcccactacgcccagctaatttttgtatttttagtagagacagggtttcaccatattggccaggctggtctcgaactcctgacctcgtgatctacctgcctcggcttcccaaagtgatttttgtatttttagtagagacggggtttcatcatattggtcaggctggtctcgaactcctgacctcaggtgatctgccctcctcggcctctgaaagtgctgggcttacaggcgtgagcaccgtgcccggactccttttttttttttttttttttgtggtggggggacaagatctcactctgtcacccaggctggatcatagctcactgtaatctcgaactcctgggctcaagcaatcctcccaagtagttggaactacaggagtattgtcaccatgcctggccaatttttattttttgtagagatggagtcttgctatgttgtccaggctgggcttgaactcctgggttcaagcaatcctcccacctcggcctcccaaagtattggaattacagatgtgagccactgtgcttgacctctttccatttttatatgccaaactaagaaagtatgttagggatagaaaagccctgctcagatatatagtctgggacattttgtggagaaatgcatcgaccttcaatttgtccctcaccctccctatactgactcattggtgattcccaaagttaggtgtcaggctttgaacacatgaggcaggtccttctttccttggtttaattttgtttttgtggctggttaaatttttctaattatttcggctagtattaaaaaagtgtttttcagctgggtgcagtggcctatgcctgtaatccccacagtgtgggaggctaaggcaggaggatctcttaagcccaggagttcgaccagcctgggcaacatagcaagactccatctctacaaaaataaaaataaaaattggccaggcatggtggcatacgcttgtagtcccagctacttgggaggctaaaggtgggaggattgctggagcccaggaggttgaggctgcagtgagttgtgattgtgccactgcactccaacctgggctaacagagcaagaccttgtcttaaaaaataaaaagtgttcttttctgaatctacctggctggtgttggggagcagcaacttcggtttcctcatcagcagaatggggtgatgatacctacctcgctgggctcctgtgggattcgagctgatgcatgctcagaggagcatccagtgtcctccctgtgtccaggaggagggcacactggagatgctcaccaatgagtatctgtctctctccttactcactgggccctcttggtagctcccagagcctcctgcccaccttatacccagctgcccagtggggagggagagctggaaccaacctgaatgtgtgagggtctgggtgtttggtggagctggggttggggctggcttggtgatgagtgtatttcctgtcactttcaggagaaagtggatgaggaggggctgaagaagctgatgggcagcctggatgagaacagtgaccagcaggtggacttccaggagtatgctgttttcctggcactcatcactgtcatgtgcaatgacttcttccagggctgcccagaccgaccctgaagcagaactcttgacttcctgccatggatctcttgggcccaggactgttgatgcctttgagttttgtattcaataaactttttttgtctgttga

SEQ ID NO: 22 - DNA, 2011 bp, Homo sapiens
  misc_feature: B-Actin       >gi|28337|emb|Y00474.1|HSACTBPR Human beta-actin gene 5′-flanking region, CpG Island 1656 to 1955
gagctctgtctcttggccagctgaatggaggcccagcggcaacacaggtcctgcctggggatcaggtctgctctgcaccccaccttgctgcctggagccgcccacctgacaacctctcatccctgctctgtagatccggtcccatccccactgcccaccccacccccccagcactccacccagttcaacgttccacgaacccccagaaccagccctcatcaacaggcagcaagaagggccccccgcccatcgccccacaacgccagccgggtgaactgtagcgttggcaggtcctgaggcagctgaaagatacaaggccagggacaggacagtcccatccccaggaggcagggagtatacaggctggggaagtttgcccttgcgtggggtggtgatggaggaggctcagcaagtcttctggactgtgaacctgtgtctgccactgtgtgctgggtggtggtcatctttcccaccaggctgtggcctctgcaaccttcaagggaggagcaggtcccattggctgagcacagccttgtacgtgaactgaacaagcagcctccttcctggccacaggttccatgtccttatatggactcatctttgcctattgcgacacacactcaatgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggcctggtcttggctgggcgtgactgttaccctcaaaagcaggcagctccagggtaaaaggtgccctgccctgtagagcccacttccttcccagggctgcggctgggtaggtttgtagccttcatcacgggccacctccagccactggaccgctggcccctgccctgtcctggggagtgtggtcctgcgactctaatggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtcggcgaagccggtgagtgagcggcgcggggccaatcgcgtgcgccgttccgaaagttgccttttatggctcgagcggccgcggcggcgccctataaaacccagcggcgcgacgcgccacc

SEQ ID NO: 23 - DNA, 1792 bp, Homo sapiens
  misc_feature: >gi|5016088|ref|NM_001101.2| Homo sapiens actin, beta (ACTB), mRNA
cgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcattgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttccccctttttgtcccccaacttgagatgtatgaaggcttttggtctccctgggagtgggtggaggcagccagggcttacctgtacactgacttgagaccagttgaataaaagtgcacacctt

SEQ ID NO: 24 - DNA, 7273 bp, Homo sapiens
  misc_feature: PTGS2       >gi|3282785|gb|AF044206.1| Homo sapiens cyclooxygenase (COX-2) gene, promoter and exon 1
ggtacccaggctggagtgcactggtgtgatcatagctcactaacctcgaactcctgggcttaggcaatcctcttgccttggcctcccaaagtgccaggattacaggcatgagccaccacagtggagctctcaattctgatactaataatttgtgtcttctctttttttccttagcctgactagagtaattaactttatgtcttttaaaagaaccacctttttggttttacccattttcttttttgattttctgtttttgatttgattgatatctactctaattttttattatttcttttcctctgcttactttgaatttaattacttttcttttttgtagtctcctaaaatagaagcttatattattgattttagatctttcttcttttctattacagcactcaatgctataaatttccctctaagcattgctttcactgcatcctacaatatttcaactctattgttatttagctcaaaagaggttcttaatttctattgggatttctctttgacccatgtgttattcagaagtgttccgtgtgatctccaaatatttgggagtttttcagctatctttctattaatcatttcttgtttaattctattgtggcctgagagcatatattgtatgatttatattcttgtaaatgtgttaaggtgtgtcttatggtgcagaatgcggtttatcttgctatatgttccttagagaataatgtatgttctgctgttattggataaagtagtctatagatgtcagttacatctcgttgattaatggtgctgttgagttcagctatgtcctaaatgattttctgtctgctgtatctgtctatttctgacacaaggctgttgaagtctccaaccataataatgaattaatctatttttctttgcagttttatcaattttgtcttatatatattgatgctccattgtttggcacatacacattaagaattgttatgtcttcttggagaatttacctttccataacatgtaacatttccctttattcctgataatttttcttgctcaaaagtttgccctgttggaaattaccagaactactctggctttatttgattagtgttagcatgctctctctttctctattcttacacttttaatgtatacttgactttgtatttaaagtggggttcttatagaaaacatatacttggtagggtgggaagtaaaataaaaagaaatacttgggtattggtttgatccactctaacaatctctatgttttaattgatgtatttagaccattgatacttatttttttatcctcatccctgtgattacccagagagctgcttaaattgattattgatatagacaaattaataattaatatctaccgtttgttactgttttctatttttcattgcccttactttctgctcctattttttgctcctttttctgttaatttaggttttgagttattttatatcattctattttctctcccttctcagcatatgaattatctttctttttgacttttttagtggctgccctgaaggttgcaatgtacatttacaaccagtcccaatctcctttcaaaaaacacaatactgtttcatggctagtgcaagtacctaataataagaagtcactcctaatttctttctctcattctttgtatctttactgttattcatttcacttgtacataagctgtaatctttcaatacattattgctattattatttcaaaacatgttatctattatatctatttaaaataagaaaaataggccaggtgcagtggcttactcatgtaatcccagcactttgggagaccgatggattgctagagctcaggaattcgagaccagcctgggcaacatagtgaaaccctgtctctactaaaaatacaaaaaaaaaaattgctgggcatggtggcatgggcctgtggtcacagctactcgggaggctgaggtgagaggattgcttgagcctgggaggcagaggttgcagtgaaccaaaatcaagctactgcactccagcctaagtgacagagtgagaccctgtctcaaaaaaaaaatgaaagaattatttttatttatcttcacttatttcttctctaatgctctttgtttctttagtatgtagatccaagtttctaacctgtatcatttttcttatctcaataacttcttttaacatttctcacaaagcagatctactggccacagaatgcctcaattttcatttgtctgagaaaaccttatttctccttcacttttgaaagataattttgtagggtacagaattctaggttgtaggttttttcccctcaaagtgaaatatttcattccactcttttcttctttgtatggtatctgagaagaagtcagatgtaattcttatcattattacttaaaagattgcttctgttcctttctctcttctccttcccttctttccttctctgtatattacaccttttatagttgccccatatttcttagatattatgttttggttttcttctgtgtttttttctttgattctcagttttagaagtctctatttatatatctgcaatcgcagggattctttcctctgccatgtccagtctactaataagcccttacagacattgttgacttctgttccagtgtttttgatctctagcatttctctgattatttcttggaattgccatctgtctacttacattaccaacctattcttgtgtgttgtcttatcatagtaattgcagttgttttaatttcataggtattgtaatttcaacatctctaccatatttgacattgattctgatgcttgctctgtcttatcaagctatgtttttgtcttttagtgtgacttctaattttttgttgaaagccaggcatgatgtactgagtgaaagaaactcaatacattgtaatgtgacgataagagttcaggggaagtgaagcattctatagtcctatagcaggtctcggccttttagtgagcctgtgcctatgaacggtgactttcaacaagtgcttttcattccactcttttcctgtccttaagtgggacaagatcactgggggggggctagaattgggtatttcccttctccaatgtagaagctaaagagagggctggagttgggtatttttcttcccctgtatggaaagctagaggcagttaaatttggatattttccttcttctaattcagttaggctgcgacaaaaatcccgacagtttaggctctaatattataaaataatttctcttgagtataggccttattaagaacactatactctgatggagctgagggggagttttctctgatattcactgcgagaacctcgtagagctccaggaagcaaaactcacaaaagtgtgggagtcttccagaatttttcctttgcagacttatctgcactgaacctccagaaattcatcaattacagttcaggttttcctacccaggtactggttttcatggaggtttctgcctgtgcatttctgctccagtaagttgttcttcttgtatggtctgtctttcaaattttttaagtagggttatgacctgtcgcctcacttctctgacagttctgagagtgttgatttttcagtttgcttagatttttacttgtttttaggatgaagtgacaatttccaagctcctccctgacatgccagatcagaaactgaaagtcctaagcctcatattctgtgcgtgggtatgttcacatcctgcctgctccagtgcccccacctcacactctctttcccttccttgtccccttgtgagatttctaggtccaatacaaagactgtgttcaactcattcaactacttggctcatctgagtattataatgaacaatcacaaaaaaaaatgaagtaaaagaaaaatccatcaaagaattgagatatttgagaaaaagaaaggagatcagtgttttataaaacttagaaatagattttttaagtgtttcttcattgacttatgtgaaaggacttttcttaatttaacaaattatgtgctttcgtttatagcctcaaaacttcttgtgtagctaagaatgggtaaataatcaggctttactaaaggactaacgtaaagatcttctgtaagtaacatttctgctactcaaggaagagataaacttcatggcataaccttgccaaagtatactaagaataaccctgacacaaagctcttttttcagccaacatgccatgaaagaaagaagacaaggggtgatctccactctctaagtgaaccactaaacccaccaaagaagaaacgagggaaatagaaagaggacccttgcctgagataatggatctgtatgtatgagtagtagaaccctgctcaaagtacaaggaagggaaaaaaaagttagtttatttggaattttggacattaagagtctttattgttcattttcttttaactcacatgaatggcttatcacttcaattaataaatatttcatttcttttcaatctatattcatgaaacaaatctgaaatgaacagtgcaacatgtgaatgtttagaacattataaaattaaacacaaaatctgtctggcaatcttcctagcatcttaggaaaaaagttgacaaaatttcaagcagcagaagggggcagtaaaactcaacagaaagctctggaagatttttaagattcttccttattttcttttcatgtagagtatttcccaacaaatttcagacgctaatagaaattttgtacaacagatccatatatttgcctaaaatagacacagaaacattgatatatgcaaacatgagagctataagttttacatgatcaaaaccttttttttatggtacacaatagtcacagtacttttccatataaaacaggtttagtggtcttaatttagtttggcacatttaatacactcccatgaccagcatcccaaatgtacctatccgttttattttattgtctcagaattgtcagttatttaataaattatgtaacttttttccttatgctcagatttgcacttctttctaaaactctgcccatccttaaagtcccagattctccttgaacttttttttttgactttccaagtacatggaactcttcactctatcctgctatataagtgacagaatttccactatgggatagatggagttcaattcctttgagtttaaaataatctaaatataattattccttatgccctgtttttccctcacttttgtatccaaatctcttttcagacaacagaacaattaatgtctgataaggaagacaatgatgatgatcacttcaaaataagcttgaattcaggattgtaatgtaaaattttagtactctctcacagtatggattctaacatggcttctaacccaaactaacattagtagctctaactataaacttcaaatttcagtagatgcaacctactcctttaaaatgaaacagaagattgaaattattaaattatcaaaaagaaaatgatccacgctcttagttgaaatttcatgtaagattccatgcaataaataggagtgccataaatggaatgatgaaatatgactagaggaggagaaaggcttcctagatgagatggaattttagtcatccgtgtctcatgaagaatcagatgtgtacactaagcaaaacagttaaaaaaaaaacctccaagtgagtctcttatttatttttttcttataagacttctacaaattgaggtacctggtgtagttttatttcaggttttatgctgtcattttcctgtaatgctaaggacttaggacataactgaattttctattttccacttcttttctggtgtgtgtgtatatatatatgtatatatacacacacacatatacatatatatatttttagtatctcaccctcacatgctcctccctgagcactacccatgatagatgttaaacaaaagcaaagatgaaattccaactgttaaaatctcccttccatctaattaattcctcatccaactatgttccaaaacgagaatagaaaattagccccaataagcccaggcaactgaaaagtaaatgctatgttgtactttgatccatggtcacaactcataatcttggaaaagtggacagaaaagacaaaagagtgaactttaaaactcgaatttattttaccagtatctcctatgaagggctagtaaccaaaataatccacgcatcagggagagaaatgccttaaggcatacgttttggacatttagcgtccctgcaaattctggccatcgccgcttcctttgtccatcagaaggcaggaaactttatattggtgacccgtggagctcacattaactatttacagggtaactgcttaggaccagtattatgaggagaatttacctttcccgcctctctttccaagaaacaaggagggggtgaaggtacggagaacagtatttcttctgttgaaagcaacttagctacaaagataaattacagctatgtacactgaaggtagctatttcattccacaaaataagagttttttaaaaagctatgtatgtatgtcctgcatatagagcagatatacagcctattaagcgtcgtcactaaaacataaaacatgtcagcctttcttaaccttactcgccccagtctgtcccgacgtgacttcctcgaccctctaaagacgtacagaccagacacggcggcggcggcgggagaggggattccctgcgcccccggacctcagggccgctcagattcctggagaggaagccaagtgtccttctgccctcccccggtatcccatccaaggcgatcagtccagaactggctctcggaagcgctcgggcaaagactgcgaagaagaaaagacatctggcggaaacctgtgcgcctggggcggtggaactcggggaggagagggagggatcagacaggagagtggggactaccccctctgctcccaaattggggcagcttcctgggtttccgattttctcatttccgtgggtaaaaaaccctgcccccaccgggcttacgcaatttttttaaggggagaggagggaaaaatttgtggggggtacgaaaaggcggaaagaaacagtcatttcgtcacatgggcttggttttcagtcttataaaaaggaaggttctctcggttagcgaccaattgtcatacgacttgcagtgagcgtcaggagcacgtccaggaactcctcagcagcgcctccttcagctccacagccagacgccctcagacagcaaagcctaccccccgcgccgcgccctgcccgaagctt

SEQ ID NO: 25 - DNA, 4465 bp, Homo sapiens
  misc_feature: >gi|4506264|ref|NM_000963.1| Homo sapiens prostaglandin-endoperoxide synthase 2 (prostaglandin G/H synthase and cyclooxygenase) (PTGS2), mRNA
caattgtcatacgacttgcagtgagcgtcaggagcacgtccaggaactcctcagcagcgcctccttcagctccacagccagacgccctcagacagcaaagcctacccccgcgccgcgccctgcccgccgctcggatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtcttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactatcacaagatggcaaaatgctgaaagtttttacactgtcgatgtttccaatgcatcttccatgatgcattagaagtaactaatgtttgaaattttaaagtacttttggttatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagatgaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatctcaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataacgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagttttaagatcagagttcactttctttggactctgcctatattttcttacctgaacttttgcaagttttcaggtaaacctcagctcaggactgctatttagctcctcttaagaagattaaaagagaaaaaaaaaggcccttttaaaaatagtatacacttattttaagtgaaaagcagagaattttatttatagctaattttagctatctgtaaccaagatggatgcaaagaggctagtgcctcagagagaactgtacggggtttgtgactggaaaaagttacgttcccattctaattaatgccctttcttatttaaaaacaaaaccaaatgatatctaagtagttctcagcaataataataatgacgataatacttcttttccacatctcattgtcactgacatttaatggtactgtatattacttaatttattgaagattattatttatgtcttattaggacactatggttataaactgtgtttaagcctacaatcattgatttttttttgttatgtcacaatcagtatattttctttggggttacctctctgaatattatgtaaacaatccaaagaaatgattgtattaagatttgtgaataaatttttagaaatctgattggcatattgagatatttaaggttgaatgtttgtccttaggataggcctatgtgctagcccacaaagaatattgtctcattagcctgaatgtgccataagactgaccttttaaaatgttttgagggatctgtggatgcttcgttaatttgttcagccacaatttattgagaaaatattctgtgtcaagcactgtgggttttaatatttttaaatcaaacgctgattacagataatagtatttatataaataattgaaaaaaattttcttttgggaagagggagaaaatgaaataaatatcattaaagataactcaggagaatcttctttacaattttacgtttagaatgtttaaggttaagaaagaaatagtcaatatgcttgtataaaacactgttcactgttttttttaaaaaaaaaacttgatttgttattaacattgatctgctgacaaaacctgggaatttgggttgtgtatgcgaatgtttcagtgcctcagacaaatgtgtatttaacttatgtaaaagataagtctggaaataaatgtctgtttatttttgtactattta

(19) United States
(12) Patent Application Publication
(10) Pub. No.: US 2008/0254455 A1
(43) Pub. Date: Oct. 16, 2008

(54) DETECTING PROSTATE CANCER

(76) Inventors: Haiying Wang, Bridgewater, NJ (US); Jonathan F. Baden, Bridgewater, NJ (US); Tatiana Vener, Sterling, NJ (US); Dondapati Chowdary, Princeton Junction, NJ (US); Abhijit Mazumder, Basking Ridge, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/734,763

(22) Filed: Apr. 12, 2007

(51) Int. Cl. C12Q 1/68; C12P 19/34; G01N 33/50

(52) U.S. Cl. 435/6; 436/64

(57) ABSTRACT

Methods and kits for detecting prostate cancer in urine samples include detecting the methylation status of various genes.

### BACKGROUND OF THE INVENTION

This invention relates to the interrogation of methylated genes in concert with other diagnostic methods and kits for use with these methods.

In higher order eukaryotes DNA is methylated only at cytosines located 5′ to guanosine in the CpG dinucleotide. This modification has important regulatory effects on gene expression, especially when it involves CpG rich areas (CpG islands) located in gene promoter regions. Aberrant methylation of normally unmethylated CpG islands is a frequent event in immortalized and transformed cells and has been associated with transcriptional inactivation of certain tumor suppressor genes or genes otherwise associated with the amelioration of certain human cancers.

A number of potential methylation markers have recently been disclosed. Glutathione S-transferases (GSTs) are exemplary proteins in which the methylation status of the genes that express them can have important prognostic and diagnostic value for prostate cancer. The proteins catalyze intracellular detoxification reactions, including the inactivation of electrophilic carcinogens, by conjugating chemically-reactive electrophiles to glutathione (C. B. Pickett, et al., Annu. Rev. Blocbern., 58:743, 1989; B. Coles, et al., CRC Crit. Rev. Biochem. Mol. Biol., 25:47, 1990; T. H. Rushmore, et al., J. Biol. Chem. 268:11475, 1993). Human GSTs, encoded by several different genes at different loci, have been classified into four families referred to as alpha, mu, pi, and theta (B. Mannervik, et al., Biochem. J., 282:305, 1992). Decreased GSTP1 expression resulting from epigenetic changes is often related to prostate and hepatic cancers.

The S100 proteins are calcium-binding proteins that are implicated in, among other things, tumerigenesis. The family includes S100A2, S100A4, S100A5, S100A6, S100A8, S100A9, and S100A11, which have all been shown to bear some relationship to tumor development though precisely what that role is has not been clear. S100A6 (calcylin) expression appears to fall off in prostate cancer development. S100A2 has been shown to exhibit lessened expression in breast, lung, and prostate cancer as well. This is believed to be due to hypermethylation of the gene promoter but the picture is not clear since hypermethylation is also seen in non-malignant prostate epithelium and BPH.

Sampling and sample preparation are important factors in epigenetic testing. Every sample source has its issues. Even biopsy samples taken directly from the affected tissue are known to present the possibility of false negative results due to uneven distribution of affected cells. Urine is a desirable sample because it can be obtained less invasively than many other potential samples. The number and concentration of prostate cancer cells shed into urine can be extremely variable depending on a host of factors such as when the urine is collected, whether it is collected pursuant to prostate massage, and the presence and effect of nucleases and reagents and methods for minimizing their effect.

While some have proposed prostate cancer testing on urine samples, actually producing such a test has proven difficult. First, it is presumed that the basis for such a test is the shedding of cancer cells from the tumor or lesion into the urinary system. Little is actually known about this process. It also seems likely that analyte concentrations could vary much more dramatically than in other samples such as tissue biopsy and even serum samples depending on a wide range of physiological and environmental factors such as the degree to which the patient is hydrated. The stability of the analyte in the matrix is also not well understood in light of the presence of nucleases and a wide variety of other substances that can affect nucleic acids. Sample preparation for a number of other urine assays use spun down samples referred to as sediments. Whether this makes sense for methylation markers cannot be supposed a priori.

Preparation of the patient and pretreatment options are also not well understood. Digital rectal examinations (DRE) are standard diagnostic procedures for determining prostate health in which the physician notes anatomical abnormalities. In the past the outcome of the DRE would be used to determine whether a biopsy or other diagnostic or therapeutic procedure would be necessary. Whether and to what extent procedures such as the DRE or related prostate massage causes cells to slough so that they would then be detected in the subsequent diagnostic procedure was unclear. Procedurally, DRE and digital rectal massage and the time in which they are performed can differ greatly further adding to the list of unknowns in this area.

### SUMMARY OF THE INVENTION

In one aspect of the invention, a method for characterizing prostate cancer in a patient comprises assaying GSTP1 methylation and one or more control genes in urine within three days of its collection. The assay is considered positive for prostate cancer if the degree of methylation of the GSTP1 exceeds a pre-determined value and is considered negative for prostate cancer if the pre-determined value is not exceeded.

In another aspect of the invention, a method for characterizing prostate cancer in a patient comprises assaying GSTP1 methylation, one or more control genes, and the S100 gene in urine within three days of its collection. A normalized value of GSTP1 is determined by comparison of the GSTP1 methylation assay value to that of the S100 methylation assay value. The assay is considered positive for prostate cancer if the normalized methylation assay value exceeds a pre-determined value and is considered negative for prostate cancer if the pre-determined value is not exceeded.

In yet another aspect of the invention, a method for characterizing prostate cancer in a patient comprises assaying GSTP1 methylation and one or more control genes conducted as a nested PCR reaction. The assay is considered positive for prostate cancer if the degree of methylation of the GSTP1 exceeds a pre-determined value and is considered negative for prostate cancer if the pre-determined value is not exceeded.

In yet another aspect of the invention, methylation of the following panels of genes is detected:

a. GSTP1, APC.

b. GSTP1, APC, S100A2.

c. GSTP1, RARβ2.

d. GSTP1, RARβ2, S100A2.

The panels may also include control genes.

In yet another aspect of the invention, methylation status is determined via quantitative real time PCR.

In yet another aspect, the invention is a kit useful for the detection of a methylated nucleic acid. The kit includes one or more containers; a first container containing a reagent that modifies unmethylated cytosine and a second container containing a reagent that primes amplification of CpG-containing nucleic acid, wherein the reagent distinguishes between modified methylated and nonmethylated nucleic acid. The kit contains instructions to conduct the assay on patients suspected of having prostate cancer.

In yet another aspect of the invention, the kit includes a reaction vessel having separate components into which primers are initially stored and wherein during use of the kit, the primers are exuded into a reaction chamber according to a set sequence such that methylation status can be properly assessed. The primers can be for conducting nested amplification reactions.

### DETAILED DESCRIPTION OF THE INVENTION

The urine-based assay of this invention is preferably conducted on patients who have had a PSA assay with ambiguous or difficult to determine results (most preferably 2.5-4.0 ng/ml). A negative result using the assay of this invention (in the absence of other clinical indicia) can spare the patient of more invasive testing such as with a biopsy procedure. Thus, viewed most inclusively, one method according to the invention involves first conducting a PSA test in a patient and then conducting the assay described more fully below on those patients having a PSA level assayed at 2.5-4.0 ng/ml.

The assays of the invention detect hypermethylation of nucleic acids that correspond to particular genes whose methylation status correlates with prostate cancer. A nucleic acid corresponds to a gene whose methylation status correlates with prostate cancer when methylation status of such a gene provides information about prostate cancer and the sequence is a coding portion of the gene or its complement, a representative portion of the gene or its complement, a promoter or regulatory sequence for the gene or its complement, a sequence that indicates the presence of the gene or its complement, or the full length sequence of the gene or its complement. Such nucleic acids are referred to as Markers in this specification. Markers correspond to the following genes only: GSTP1 (Seq. ID. No. 17), APC (Promoter=Seq. ID. No. 18, Gene=Seq. ID. No. 19), RARβ2 (Seq. ID No. 20), S100A2 (Seq. ID. No. 21). Other sequences of interest include constitutive genes useful as assay controls such as beta-Actin (Seq. ID. No. 22 and 23) and PTGS2 (Promoter=Seq. ID. No. 24, Gene=Seq. ID. No. 25).

Assays for detecting hypermethylation include such techniques as MSP and restriction endonuclease analysis. The promoter region is a particularly noteworthy target for detecting such hypermethylation analysis. Sequence analysis of the promoter region of GSTP1 shows that nearly 72% of the nucleotides are CG and about 10% are CpG dinucleotides.

The invention includes determining the methylation status of certain regions of the Markers in urine or urethral washes and in which the DNA associated with prostate cancer is amplified and detected. Since a decreased level of the protein encoded by the Marker (i.e., less transcription) is often the result of hypermethylation of a particular region such as the promoter, it is desirable to determine whether such regions are hypermethylated. This is seen most demonstrably in the case of the GSTP1 gene and in the panels indicated in the Summary of the Invention. A nucleic acid probe or reporter specific for certain Marker regions is used to detect the presence of methylated regions of the Marker gene. Hypermethylated regions are those that are methylated to a statistically significant greater degree in samples from diseased tissue as compared to normal tissue.

As noted above, urine is the matrix in which the assays of this invention are conducted. Most preferably, it is collected after prostate massage and stored at 4C until it can be sedimented. It is most preferably spun down within 4 hours.

Prostatic massage, when conducted in conjunction with the methylation analyses of the invention, is best conducted as follows: the gland is pressed firmly enough to depress the surface from the base to the apex and from the lateral to the median line for each lobe to ensure the release of sufficient number of prostate cells. It is most preferred that this massage procedure is conducted for 20 seconds or less.

Some of the primers/probes or reporter reagents of the invention are used to detect methylation of expression control sequences of the Marker genes. These are nucleic acid sequences that regulate the transcription and, in some cases, translation of the nucleic acid sequence. Thus, expression control sequences can include sequences involved with promoters, enhancers, transcription terminators, start codons (i.e., ATG), splicing signals for introns, maintenance of the correct reading frame of that gene to permit proper translation of the mRNA, and stop codons.

The GSTP1 promoter is the most preferred Marker. It is a polynucleotide sequence that can direct transcription of the gene to produce a glutathione-s-transferase protein. The promoter region is located upstream, or 5′ to the structural gene. It may include elements which are sufficient to render promoter-dependent gene expression controllable for cell-type specific, tissue-specific, or inducible by external signals or agents; such elements may be located in the 5′ or 3′ regions of the of the polynucleotide sequence.

One method of the invention includes contacting a target cell containing a Marker with a reagent that binds to the nucleic acid. The target cell component is a nucleic acid such as DNA extracted from urine by cell lysis and purification (column or solution based) yielding pure DNA that is devoid of proteins. The reagents include components that prime and probe PCR or MSP reactions and detect the target sequence. These reagents can include priming sequences combined with or bonded to their own reporter segments such as those referred to as Scorpion reagents or Scorpion reporters and described in U.S. Pat. Nos. 6,326,145 and 6,270,967 to Whitcombe et. al. (incorporated herein by reference in their entirety). Though they are not the same, the terms “primers” and “priming sequences” may be used in this specification to refer to molecules or portions of molecules that prime the amplification of nucleic acid sequences.

One sensitive method of detecting methylation patterns involves combining the use of methylation-sensitive enzymes and the polymerase chain reaction (PCR). After digestion of DNA with the enzyme, PCR will amplify from primers flanking the restriction site only if DNA cleavage was prevented by methylation. The PCR primers of the invention are designed to target the promoter and transcription region that lies approximately between−71 and+59 bp according to genomic positioning number of M24485 (Genbank) from the transcription start site of GSTP1.

The method of the invention can also include contacting a nucleic acid-containing specimen with an agent that modifies unmethylated cytosine; amplifying the CpG-containing nucleic acid in the specimen by means of CpG-specific oligonucleotide primers; and detecting the methylated nucleic acid. The preferred modification is the conversion of unmethylated cytosines to another nucleotide that will distinguish the unmethylated from the methylated cytosine. Preferably, the agent modifies unmethylated cytosine to uracil and is sodium bisulfite, however, other agents that modify unmethylated cytosine, but not methylated cytosine can also be used. Sodium bisulfite (NaHSO₃) modification is most preferred and reacts readily with the 5,6-double bond of cytosine, but poorly with methylated cytosine. Cytosine reacts with the bisulfite ion to form a sulfonated cytosine reaction intermediate susceptible to deamination, giving rise to a sulfonated uracil. The sulfonate group can be removed under alkaline conditions, resulting in the formation of uracil. Uracil is recognized as a thymine by Taq polymerase and therefore upon PCR, the resultant product contains cytosine only at the position where 5-methylcytosine occurs in the starting template. Scorpion reporters and reagents and other detection systems similarly distinguish modified from unmodified species treated in this manner.

The primers used in the invention for amplification of a CpG-containing nucleic acid in the specimen, after modification (e.g., with bisulfite), specifically distinguish between untreated DNA, methylated, and non-methylated DNA. In methylation specific PCR (MSPCR), primers or priming sequences for the non-methylated DNA preferably have a T in the 3″ CG pair to distinguish it from the C retained in methylated DNA, and the complement is designed for the antisense primer. MSP primers or priming sequences for non-methylated DNA usually contain relatively few Cs or Gs in the sequence since the Cs will be absent in the sense primer and the Gs absent in the antisense primer (C becomes modified to U (uracil) which is amplified as T (thymidine) in the amplification product).

The primers of the invention are oligonucleotides of sufficient length and appropriate sequence so as to provide specific initiation of polymerization on a significant number of nucleic acids in the polymorphic locus. When exposed to appropriate probes or reporters, the sequences that are amplified reveal methylation status and thus diagnostic information.

Preferred primers are most preferably eight or more deoxyribonucleotides or ribonucleotides capable of initiating synthesis of a primer extension product, which is substantially complementary to a polymorphic locus strand. Environmental conditions conducive to synthesis include the presence of nucleoside triphosphates and an agent for polymerization, such as DNA polymerase, and a suitable temperature and pH. The priming segment of the primer or priming sequence is preferably single stranded for maximum efficiency in amplification, but may be double stranded. If double stranded, the primer is first treated to separate its strands before being used to prepare extension products. The primer must be sufficiently long to prime the synthesis of extension products in the presence of the inducing agent for polymerization. The exact length of primer will depend on factors such as temperature, buffer, cations, and nucleotide composition. The oligonucleotide primers most preferably contain about 12-20 nucleotides although they may contain more or fewer nucleotides, preferably according to well known design guidelines or rules.

Primers are designed to be substantially complementary to each strand of the genomic locus to be amplified and include the appropriate G or C nucleotides as discussed above.

This means that the primers must be sufficiently complementary to hybridize with their respective strands under conditions that allow the agent for polymerization to perform. In other words, the primers should have sufficient complementarity with the 5′ and 3′ flanking sequence(s) to hybridize and permit amplification of the genomic locus.

The primers are employed in the amplification process. That is, reactions (preferably, an enzymatic chain reaction) that produce greater quantities of target locus relative to the number of reaction steps involved. In a most preferred embodiment, the reaction produces exponentially greater quantities of the target locus. Reactions such as these include the PCR reaction. Typically, one primer is complementary to the negative (−) strand of the locus and the other is complementary to the positive (+) strand. Annealing the primers to denatured nucleic acid followed by extension with an enzyme, such as the large fragment of DNA Polymerase I (Klenow) and nucleotides, results in newly synthesized+and−strands containing the target locus sequence. The product of the chain reaction is a discrete nucleic acid duplex with termini corresponding to the ends of the specific primers employed.

The primers may be prepared using any suitable method, such as conventional phosphotriester and phosphodiester methods including automated methods. In one such automated embodiment, diethylphosphoramidites are used as starting materials and may be synthesized as described by Beaucage, et at. (Tetrahedron Letters, 22:1859-1862, 1981). A method for synthesizing oligonucleotides on a modified solid support is described in U.S. Pat. No. 4,458,066.

Any nucleic acid specimen taken from urine or urethral wash, in purified or non-purified form, can be utilized as the starting nucleic acid or acids, provided it contains, or is suspected of containing, the specific nucleic acid sequence containing the target locus (e.g., CpG). Thus, the process may employ, for example, DNA or RNA, including messenger RNA. The DNA or RNA may be single stranded or double stranded. In the event that RNA is to be used as a template, enzymes, and/or conditions optimal for reverse transcribing the template to DNA would be utilized. In addition, a DNA-RNA hybrid containing one strand of each may be utilized. A mixture of nucleic acids may also be employed, or the nucleic acids produced in a previous amplification reaction herein, using the same or different primers may be so utilized. The specific nucleic acid sequence to be amplified, i.e., the target locus, may be a fraction of a larger molecule or can be present initially as a discrete molecule so that the specific sequence constitutes the entire nucleic acid.

If the extracted sample is impure, it may be treated before amplification with an amount of a reagent effective to open the cells, fluids, tissues, or animal cell membranes of the sample, and to expose and/or separate the strand(s) of the nucleic acid(s). This lysing and nucleic acid denaturing step to expose and separate the strands will allow amplification to occur much more readily.

Where the target nucleic acid sequence of the sample contains two strands, it is necessary to separate the strands of the nucleic acid before it can be used as the template. Strand separation can be effected either as a separate step or simultaneously with the synthesis of the primer extension products. This strand separation can be accomplished using various suitable denaturing conditions, including physical, chemical or enzymatic means. One physical method of separating nucleic acid strands involves heating the nucleic acid until it is denatured. Typical heat denaturation may involve temperatures ranging from about 80 to 105° C. for up to 10 minutes. Strand separation may also be induced by an enzyme from the class of enzymes known as helicases or by the enzyme RecA, which has helicase activity, and in the presence of riboATP, is known to denature DNA. Reaction conditions that are suitable for strand separation of nucleic acids using helicases are described by Kuhn Hoffmann-Berling (CSH-Quantitative Biology, 43:63, 1978). Techniques for using RecA are reviewed in C. Radding (Ann. Rev. Genetics, 16:405-437, 1982). Refinements of these techniques are now also well known.

When complementary strands of nucleic acid or acids are separated, regardless of whether the nucleic acid was originally double or single stranded, the separated strands are ready to be used as a template for the synthesis of additional nucleic acid strands. This synthesis is performed under conditions allowing hybridization of primers to templates to occur. Generally synthesis occurs in a buffered aqueous solution, preferably at a pH of 7-9, most preferably about 8. A molar excess (for genomic nucleic acid, usually about 10⁸:1, primer:template) of the two oligonucleotide primers is preferably added to the buffer containing the separated template strands. The amount of complementary strand may not be known if the process of the invention is used for diagnostic applications, so the amount of primer relative to the amount of complementary strand cannot always be determined with certainty. As a practical matter, however, the amount of primer added will generally be in molar excess over the amount of complementary strand (template) when the sequence to be amplified is contained in a mixture of complicated long-chain nucleic acid strands. A large molar excess is preferred to improve the efficiency of the process.

The deoxyribonucleoside triphosphates dATP, dCTP, dGTP, and dTTP are added to the synthesis mixture, either separately or together with the primers, in adequate amounts and the resulting solution is heated to about 90-100° C. for up to 10 minutes, preferably from 1 to 4 minutes. After this heating period, the solution is allowed to cool to room temperature, which is preferable for the primer hybridization. To the cooled mixture is added an appropriate agent for effecting the primer extension reaction (the “agent for polymerization”), and the reaction is allowed to occur under conditions known in the art. The agent for polymerization may also be added together with the other reagents if it is heat stable. This synthesis (or amplification) reaction may occur at room temperature up to a temperature at which the agent for polymerization no longer functions.

The agent for polymerization may be any compound or system that will function to accomplish the synthesis of primer extension products, preferably enzymes. Suitable enzymes for this purpose include, for example, E. coli DNA polymerase 1, Klenow fragment of E. coli DNA polymerase I, T4 DNA polymerase, other available DNA polymerases, polymerase mutants, reverse transcriptase, and other enzymes, including heat-stable enzymes (e.g., those enzymes which perform primer extension after being subjected to temperatures sufficiently elevated to cause denaturating). A preferred agent is Taq polymerase. Suitable enzymes will facilitate combination of the nucleotides in the proper manner to form the primer extension products complementary to each locus nucleic acid strand. Generally, the synthesis will be initiated at the 3′ end of each primer and proceed in the 5′ direction along the template strand, until synthesis terminates, producing molecules of different lengths. There may be agents for polymerization, however, which initiate synthesis at the 5′ end and proceed in the other direction, using the same process as described above.

Most preferably, the method of amplifying is by PCR. Alternative methods of amplification can also be employed as long as the methylated and non-methylated loci amplified by PCR using the primers of the invention is similarly amplified by the alternative means. In one such most preferred embodiment, the assay is conducted as a nested PCR. In nested PCR methods, two or more staged polymerase chain reactions are undertaken. In a first-stage polymerase chain reaction, a pair of outer oligonucleotide primers, consisting of an upper and a lower primer that flank a particular first target nucleotide sequence in the 5′ and 3′ position, respectively, are used to amplify that first sequence. In subsequent stages, a second set of inner or nested oligonucleotide primers, also consisting of an upper and a lower primer, are used to amplify a smaller second target nucleotide sequence that is contained within the first target nucleotide sequence. The upper and lower inner primers flank the second target nucleotide sequence in the 5′ and 3′ positions, respectively. Flanking primers are complementary to segments on the 3′-end portions of the double-stranded target nucleotide sequence that is amplified during the PCR process.

The first nucleotide sequence within the region of the gene targeted for amplification in the first-stage polymerase chain reaction is flanked by an upper primer in the 5′ upstream position and a lower primer in the 3′ downstream position. The first targeted nucleotide sequence, and hence the amplification product of the first-stage polymerase chain reaction, has a predicted base-pair length, which is determined by the base-pair distance between the 5′ upstream and 3′ downstream hybridization positions of the upper and lower primers, respectively, of the outer primer pair.

At the end of the first-stage polymerase chain reaction, an aliquot of the resulting mixture is carried over into a second-stage polymerase chain reaction. This is preferably conducted within a sealed or closed vessel automatically such as with the “SMART CAP” device from Cepheid. In this second-stage reaction, the products of the first-stage reaction are combined with specific inner or nested primers. These inner primers are derived from nucleotide sequences within the first targeted nucleotide sequence and flank a second, smaller targeted nucleotide sequence contained within the first targeted nucleotide sequence. This mixture is subjected to initial denaturation, annealing, and extension steps, followed by thermocycling as before to allow for repeated denaturation, annealing, and extension or replication of the second targeted nucleotide sequence. This second targeted nucleotide sequence is flanked by an upper primer in the 5′ upstream position and a lower primer in the 3′ downstream position. The second targeted nucleotide sequence, and hence the amplification product of the second-stage PCR, also has a predicted base-pair length, which is determined by the base-pair distance between the 5′ upstream and 3′ downstream hybridization positions of the upper and lower primers, respectively, of the inner primer pair.

The amplified products are preferably identified as methylated or non-methylated with a probe or reporter specific to the product as described in U.S. Pat. No. 4,683,195 to Mullis et. al., incorporated herein by reference in its entirety. Advances in the field of probes and reporters for detecting polynucleotides are well known to those skilled in the art. Optionally, the methylation pattern of the nucleic acid can be confirmed by other techniques such as restriction enzyme digestion and Southern blot analysis. Examples of methylation sensitive restriction endonucleases which can be used to detect 5′CpG methylation include SmaI, SacII, EagI, MspI, HpaII, BstUI and BssHII.

In another aspect of the invention a methylation ratio is used. This can be done by establishing a ratio between the amount of amplified methylated species of Marker attained and the amount of amplified reference Marker or non-methylated Marker region amplified. This is best done using quantitative real-time PCR. Ratios above an established or predetermined cutoff or threshold are considered hypermethylated and indicative of having a proliferative disorder such as cancer (prostate cancer in the case of GSTP1). Cutoffs are established according to known methods in which such methods are used for at least two sets of samples: those with known diseased conditions and those with known normal conditions. The reference Markers of the invention can also be used as internal controls. The reference Marker is preferably a gene that is constitutively expressed in the cells of the samples such as Beta Actin.

Established or predetermined values (cutoff or threshold values) are also established and used in methods according to the invention in which a ratio is not used. In this case, the cutoff value is established with respect to the amount or degree of methylation relative to some baseline value such as the amount or degree of methylation in normal samples or in samples in which the cancer is clinically insignificant (is known not to progress to clinically relevant states or is not aggressive). These cutoffs are established according to well-known methods as in the case of their use in methods based on a methylation ratio.

In the most preferred embodiment of the invention, GSTP1 methylation values obtained by MSP or other suitable methods are normalized with S100A2 methylation values determined using the same method. The normalized value is obtained by subtracting the S100A2 assay value from that of the GSTP1 value as shown in Example 7. Other normalization methods can also be used such as generation of a methylation ratio (obtained by converting the Ct value to a copy number for the gene of interest and dividing that copy number by the copy number for beta-actin, obtained in the same manner). When using a normalized value, the cutoff value is determined by first generating a training set in which the cutoff generates optimal sensitivity and specificity and then validating the cutoff in an independent validation set.

The inventive methods and kits can include steps and reagents for multiplexing. That is, more than one Marker can be assayed at a time. But only the following Markers are assayed as part of this invention GSTP1, RAR-β2, APC, and S100A2 along with internal controls such as β-Actin.

Since a decreased level of transcription of the gene associated with the Marker is often the result of hypermethylation of the polynucleotide sequence and/or particular elements of the expression control sequences (e.g., the promoter sequence), primers prepared to match those sequences were prepared. Accordingly, the invention provides methods of detecting or diagnosing a cell proliferative disorder by detecting methylation of particular areas, preferably, within the expression control or promoter region of the Markers. Probes useful for detecting methylation of these areas are useful in such diagnostic or prognostic methods.

The kits of the invention can be configured with a variety of components provided that they all contain at least one primer or probe or a detection molecule (e.g., Scorpion reporter). In one embodiment, the kit includes reagents for amplifying and detecting hypermethylated Marker segments. Optionally, the kit includes sample preparation reagents and/or articles (e.g., tubes) to extract nucleic acids from samples.

In a preferred kit, reagents necessary for one-tube MSP are included such as, a corresponding PCR primer set, a thermostable DNA polymerase, such as Taq polymerase, and a suitable detection reagent(s) such as hydrolysis probe or molecular beacon. In optionally preferred kits, detection reagents are Scorpion reporters or reagents. A single dye primer or a fluorescent dye specific to double-stranded DNA such as ethidium bromide can also be used. The primers are preferably in quantities that yield high concentrations. Additional materials in the kit may include: suitable reaction tubes or vials, a barrier composition, typically a wax bead, optionally including magnesium; necessary buffers and reagents such as dNTPs; control nucleic acid (s) and/or any additional buffers, compounds, co-factors, ionic constituents, proteins and enzymes, polymers, and the like that may be used in MSP reactions. Optionally, the kits include nucleic acid extraction reagents and materials.

### EXAMPLES

### Example 1

### Sample Preparation and MSPCR

Prostate samples were obtained from patients with known clinical outcomes.

The methylation assays were conducted as follows. Genomic DNA was modified using a commercially available sodium bisulfite conversion reagent kit (Zymo Research, Orange, Calif., USA). This treatment converted all Cytosines in unmethylated DNA into Uracil, whereas in methylated DNA only cytosines not preceding guanine were converted into Uracil. All cytosines preceeding guanine (in a CpG dinucletide) remained as cytosine.

The assays are described more fully below.

a. Sedimentation: Sedimented Urine Samples were Obtained as Follows.

50-ml Falcon tubes containing the urine were centrifuged at 3000 g on VRX Sorvall centrifuge for 10 minutes at +4 degrees C. Supernatant was removed leaving ˜5 ml on top of the pellet. The tubes were then spun down again (3000 g for 5 minutes) in order to discard the remaining supernatant (using 1 ml tips). Urine sediment was then rinsed with 20 mL cold (4C) PBS, spun down again (3000 g for 5 minutes), and the residual supernatant was aspirated. Samples were then stored at −20C.

b. Cell Lysis and DNA Extraction:

The cells in the sediment were then lysed as follows. 700 μl Cell Lysis Solution was added to each sample containing a urine cell pellet. The lysate was then transferred in a 2.0 ml microfuge tube and 3 μl Proteinase K Solution (20 mg/ml) was added to the lysate, mixed by inverting 25 times, and incubated for one hour to overnight at 55° C.

Samples were cooled to room temperature by placing at 20° C. (heat block) for 10 minutes. 300 μl Protein Precipitation Solution was then added to the lysate which was then vortexed vigorously at high speed for 20 seconds The samples were placed into an ice bath for 5 minutes and centrifuged at (16000 RPM) for 5 minutes. The precipitated proteins formed a tight pellet. The supernatant was then transferred to a new 2.0 ml tube with the precipitation steps repeated.

The supernatant containing the DNA was then transferred into a clean 2.0 ml microfuge tube and centrifugation was repeated (16000 RPM for 3 minutes) with the supernatant again transferred into a clean 2.0 ml microfuge tube containing 900 gl 100% isopropanol and 2 gl Glycogen 20 mg/ml. The sample was mixed by inverting gently 50 times and kept at room temperature for at least 10-15 minutes on the rocker and then cooled to −20C. The sample was then centrifuge at 16000 RPM for 5 minutes. The DNA was then visible as a small white pellet. Supernatant was removed with the 1 ml-pipet and the sample was centrifuge at (16000 RPM) for 60 seconds. Remaining supernatant was removed with a 100 μl-pipet. 900 μl 70% ethanol was added and the tube was inverted 10 times to wash the DNA pellet followed by another centrifugation at 16000 RPM for 1 minute. Ethanol was discarded with the 1 ml-pipet followed by another centrifugation at (16000 RPM) for 60 seconds. The remaining supernatant was discarded with the 100 μl-pipet and the sample was allowed to air dry 10-15 minutes.

45 μl LoTE buffer was added to the dried samples and the DNA was rehydrated by incubating at 65° C. for 1 hour shaking at 1100 rpm and overnight at 20° C. shaking at 1100 rpm. The DNA was stored in a clearly labelled tube at −80° C.

c. Bisulfite Modification:

DNA Samples were then modified using EZ-DNA methylation kit from ZymoResearch (Cat. No D5001) as follows.

24 ml absolute Ethanol was added to the M-Wash buffer Concentrate to make the final M-Wash buffer. 5 ul of M-Dilution Buffer directly to 45 μl of the DNA sample. This mixture was mixed by pipetting up and down and then spun briefly followed by incubation at 37° C. for 15 minutes in a heat block with shaking at 1100 rpm. During the incubation, CT Conversion Reagent was prepared by adding 750 μl Baker Water and 210 μl of M-Dilution Buffer. It was then mixed by vortexing for 1 minute every 2 minutes for a total of 10 minutes. After the above incubation, 100 gl of the prepared CT Conversion Reagent (after briefly spinning) was added to each sample which was then vortexed lightly and spun briefly. The sample was then incubated at 70° C. for 3 hour with the heating block (shaking at 1100 rpm) covered with aluminum foil.

The sample was then spun down briefly and set on ice for 10 minutes. 400 μl of M-Binding buffer was added to the sample which was mixed by pipetting up and down. All the supernatant was loaded into a Zymo-Spin Column which was placed into a 2 ml collection tube. The tube was centrifuged at maximum speed for 15-30 seconds the flow-through discarded. 200 μl of M-Wash Buffer was added to the column which was centrifuge at maximum speed for 15-30 seconds again with the flow-through again discarded.

200 μl of M-Desulphonation Buffer was added to the column and let to stand at room temperature for 15 minutes followed by centrifugation at maximum speed for 15-30 seconds with the flow-through discarded. This procedure was followed three times with the last centrifugation step lasting 30 seconds. The column was then placed onto a clean 1.5 ml tube to which 50 ul of M-elution buffer was added. The columns were then let to stand for 1 min at RT followed by centrifugation at maximum speed for 1 minute to elute the DNA. The eluted DNA was labeled and stored as ‘BT modified’ at −80° C.

MSPCR assays were then set up with the following primers and probes:

Outer PCR primers

| | | |
|---|---|---|
| GSTP1_332_{—} | Seq ID No. 1 | TCGGGGATTTTAGGGCGT |
| U18 | | |
| GSTP1_513_{—} | Seq ID No. 2 | ACGAAAACTACGACGACGAAA |
| L21 | | |
| Actin_309_{—} | Seq ID No. 5 | GATATAAGGTTAGGGATAGGATAG |
| U24 | | |
| Actin_501_{—} | Seq ID No. 6 | AACCAATAAAACCTACTCCTCC |
| L22 | | |
| APC_Outer_{—} | Seq ID No. 9 | CCCTATACCCCACTACGAA |
| 692_U19 | | |
| APC_Outer_{—} | Seq ID No. 10 | GGCGGGTTGTATTAATATAGTTATA |
| 830_L25 | | |
| RARB2_Out- | Seq ID No. 13 | GGAAGTGAGTTGTTTAGAGGTAGGA |
| er_16_U25 | | |
| RARB2_Out- | Seq ID No. 14 | TCCAAACTTACTCGACCAATCCAAC |
| er_239_L25 | | |

Inner PCR Scorpion probe/primer sets

| | | |
|---|---|---|
| | | Seq |
| | | ID |
| Description | Sequence | No |

| | | |
|---|---|---|
| GSTP1 | FAM-CGCACGGCGAACTCCCGCCGACGTGCG | 3 |
| Scorpion | BHQ-HEG-TGTAGCGGTCGTCGGGGTTG | |
| GSTPi | 5′ GCCCCAATACTAAATCACGACG 3′ | 4 |
| Reverse | | |
| Primer | | |
| Actin | Q670-CCGCGCATCACCACCCCACACGCGCGG- | 7 |
| Scorpion | BHQ2-HEG-GGAGTATATAGGTTGGGGAAGTTTG | |
| Actin | 5′ AACACACAATAACAAACACAAATTCAC 3′ | 8 |
| Reverse | | |
| Primer | | |
| APC | Texas Red - | 11 |
| Scorpion | GCCGGCGGGTTTTCGACGGGCCGGC- | |
| | BHQ2-HEG-CGAACCAAAACGCTCCCCA | |
| APC Lower | GTCGGTTACGTGCGTTTATATTTAG | 12 |
| Primer | | |
| RARB2 | Q570 - CGGCGCCCGACGATACCCAAAGCGCCG- | 15 |
| Scorpion | BHQ2-HEG- AACGCGAGCGATTCGAGTAG | |
| RARB2 | CTTACAAAAAACCTTCCGAATACG | 16 |
| Lower | | |
| Primer | | |

BHQ = Black Hole Quencher reporter molecule. HEG = hexaethylene glycol

Nested PCR reactions were conducted using “SMARTCAP” tubes (Cepheid) and the “SMARTCYCLER” (Cepheid) PCR analyzer as follows.

Thawed reagents were each vortexed briefly to mix. Adequate Cepheid SmartCap PCR reaction tubes were labeled and placed it in the rack. Using a fresh pipette tip for each tube, 5 ul of the first round PCR master mix were added into each tube. Again with a fresh pipette tip for each specimen, 5 μL of specimen were added to the respective tubes which were then closed without snapping SmartCaps in place. The tubes were centrifuged for 30 seconds in the “SMARTCYCLER” centrifuge and placed in sequence in the

“SMARTCYCLER” instrument with the lid on the “SMARTCYCLER” closed instrument and the run initiated run. The cycling conditions on the Cepheid platform are indicated below (1^{st} round PCR).

Following completion of the run, the tubes were removed and a second round of PCR was set up as follows. The tube lid was opened followed by the addition of 15 ul of the second round PCR master mix into the “SMARTCAP” reservoir to the final volume of 25 ul. The spike was inserted and the lid was snapped into place. The tubes were then centrifuged for 30 seconds in the microcentrifuge with a suitable rotor. The inner PCR reaction was then run for 40 cycles under cycling conditions on the Cepheid platform as indicated below (2nd round PCR). Following completion of the run, the Cepheid tubes were removed and discarded.

The following cycling parameters were used.

First Round PCR

| Temperature | Time | | Cycles |
|---|---|---|---|
| 94 C. | 2 | min | 1 |
| 92 C. | 20 | sec | |
| 55 C. | 30 | sec | 18 |
| 70 C. | 30 | sec | |
| 70 C. | 5 | min | 1 |

Second Round PCR

| Temperature | Time | | Cycles |
|---|---|---|---|
| 95 C. | 1 | min | 1 |
| 95 C. | 20 | sec | 40 |
| 59 C. | 30 | sec | collection |

The reaction mix for a single quadruplex in the SMARTCAP tubes was prepared using the following individual components.

First round PCR (R1)

| Master Mix (MM1) | |
|---|---|
| Reagents | ul |
| DNA template (ul) | 5.00 |
| 10x Magic Buffer | 1 |
| Taq (Ab) Polymerase | 0.5 |
| 10x outer Primer Mix | 1 |
| 2.5 mM dNTPs (100 nM) | 0.4 |
| Water | 2.10 |
| Total | 10.0 |

Outer primer PM-1 final Cone: All/Actin markers-0.05 uM-0.04 uM

Second Round PCR (R2)

| Master Mix (MM2) | Pre-mix (ul w/o sample) |
|---|---|
| Reagents | ul |
| DNA template (ul) | 0.0 |
| 10x Magic Buffer | 1.5 |
| Taq (Ab) Polymerase | 1.5 |
| 25x inner primer Mix-4p | 1 |
| 25 mM dNTPs (1 mM) | 1 |
| Water | 10.0 |
| Total | 15.0 |

Inner primer final Cone: GSTP1/RARB/APC-0.4 uM/Actin-0.24 uM

The PCR Master Mixes were prepared as follows (outer primer and inner Scorpion probe/primer mixes)

10X outer primer Mix-4p-GSTP1-0.5/RARB-0.5/APC-0.5 uM/Actin-
0.4 uM
Outer primer final Concentrations: GSTP1-0.05/RARB-0.05/APC-
0.05 uM/Actin-0.04 uM
Calculations are shown for a single reaction and a batch of 200.

| Primer concentration | ul per 1 rxn | 200 |
|---|---|---|
| 100 uM GSTP1_332_U18 | 0.005 | 1 |
| 100 uM GSTP1_513_L21 | 0.005 | 1 |
| 100 uM APC_Outer_692_U19 | 0.005 | 1 |
| 100 uM APC_Outer_830_L25 | 0.005 | 1 |
| 100 uM RARB2_Outer_16_U25 | 0.005 | 1 |
| 100 uM RARB2_Outer_239_L25 | 0.005 | 1 |
| 100 uM Actin_309_U24 | 0.004 | 0.8 |
| 100 uM Actin_501_L22 | 0.004 | 0.8 |
| Water | 0.962 | 192 |
| Total | 1 | 200 |

25X inner primer/probe Mix-4p (10 uM each/6 uM actin)
Inner primer final Concentrations: GSTP1/RARB/APC-0.4 uM/Actin-
0.24 uM. Calculations are shown for a single reaction and a batch of
200.

| Primer concentration | ul per 1 rxn | 200 |
|---|---|---|
| 100 uM GSTP1_Fam_Sc_1112_L15 | 0.1 | 20 |
| 100 uM GSTPi_1151_L22 | 0.1 | 20 |
| 100 uM RARB2_M_136_AS15_Q570 | 0.1 | 20 |
| 100 uM RARB2_165_L24 | 0.1 | 20 |
| 100 uM APC_M_781_AS15_TR | 0.1 | 20 |
| 100 uM APC_804_L25 | 0.1 | 20 |
| 100 uM Actin_Q670_Sc_382_L15 (Cy5) | 0.06 | 12 |
| 100 uM Actin_425_L27 | 0.06 | 12 |
| Water | 0.28 | 56 |
| Total | 1 | 200 |

The final 25 μl reaction contents were as follows:

| Component | Final Conc in Rxn |
|---|---|
| (NH₄)₂ SO₄ | 16.6 mM |
| Tris pH 8.8 | 67 mM |
| MgCl₂ | 6.7 mM |
| B-ME | 10 mM |
| Each dNTP | 1.25 mM |
| Each Primer | 400 nM |
| Each Scorpion | 400 nM |
| Tris-HCl, pH 8.3 | 4 mM |
| KCl | 20 mM |
| EDTA | 0.02 mM |
| DTT | 0.2 mM |
| Nonidet P40 | 0.02% (v/v) |
| Tween 20 | 0.02% (v/v) |
| Glycerol | 2% (v/v) |
| TP6-25 antibody | 0.65 ug |
| Taq Polymerase | 5 U |
| Tris-HCl, pH 7.6 | 1.8 mM |
| Glycerol | 0.70% |
| ProClin 300 | 0.02% |

Data output from the “SMARTCYCLER” analyzer.

Data were analyzed through the implementation of predetermined thresholds and criteria are shown in Table 1.

TABLE 1

| | | Valid | | | | |
|---|---|---|---|---|---|---|
| | | Min | Valid Max | Bkgd Min | Bkgd Mix | Box Car |
| Ch # | Threshold | Cycle | Cycle | Cycle | Cycle | Avg. |
| FAM | 30 | 13 | 40 | 5 | 45 | 0 |
| Cy3 | 20 | 13 | 40 | 5 | 45 | 0 |
| TxR | 20 | 13 | 40 | 5 | 45 | 0 |
| Cy5 | 20 | 13 | 40 | 5 | 45 | 0 |

Results were generated and are presented as the following assay performance characteristics: % Sensitivity, Specificity and 95% confidence intervals, calculated for the combination of markers at defined Ct cutoffs for 2 in 1 PCR format. Area under the curve values were calculated based on ROC curve analysis performed with two statistical software packages. For a single marker analysis, AUC values were generated using MedCalc software and for different combinations of multiple markers, logistic regression model in S-Plus statistical software was applied.

A cut-off value was set based on the relative distribution of Ct values between the cancer and non-cancer patients. If either one of Ct values from the set of methylation markers was below the defined cutoff, the sample was considered methylated, even if Actin indicated the “no test” case. The figures show the data compared across a variety of parameters to illustrate the various embodiments of the invention.

### Example 2

### Effect of Sample Storage and Panel Identification

The procedure described in example 1 was repeated with combinations assays comprising a combination of Markers from the GSTP1, RARβ2, and APC genes. Assays were conducted within 3 days of sample collection, 5 days of sample collection, and 16 days of sample collection as indicated. Results are shown in Table 2.

TABLE 2

| | | | Days | Sensitivity | Specificity |
|---|---|---|---|---|---|
| GSTP | RARβ2 | APC | Stored | (%) | (%) |
| X | | | 16 | 31 | 96 |
| | | X | 16 | 31 | 93 |
| | X | | 16 | 36 | 86 |
| X | | X | 16 | 44 | 91 |
| X | X | | 16 | 39 | 85 |
| X | X | X | 16 | 49 | 82 |
| X | | | 5 | 35 | 96 |
| | | X | 5 | 35 | 91 |
| | X | | 5 | 40 | 85 |
| X | | X | 5 | 50 | 90 |
| X | X | | 5 | 44 | 84 |
| X | X | X | 5 | 54 | 81 |
| X | | | 3 | 36 | 91 |
| | | X | 3 | 32 | 88 |
| | X | | 3 | 29 | 93 |
| X | | X | 3 | 54 | 88 |
| X | X | | 3 | 39 | 84 |
| X | X | X | 3 | 54 | 81 |

The sample set for this example were whole (neat) urine samples and consisted of 148 samples (68 known cancers) for the 16 day set, 121 samples (52 known cancers) for the 5 day set, and 73 samples (30 known cancers) for the three day set. Surprisingly, the combination of GSTP and RARβ2 outperformed the combination of GSTP, RARβ2, and APC despite APC being a known prostate cancer marker. This two-gene combination vastly outperformed any other when samples were stored for three days or less. When all tests were considered, the positive predictive value for samples stored for 3 days was 65.9% compared to 51.35% for samples held for 5 days, and 47.22% for samples stored for 16 days. ROC curves analyses were then conducted for new data sets (N=73, known cancers=30, known non-cancers=43) using whole urine samples and using sediments prepared as described above and stored for 3 days. The area under the curve was determined for individual Markers. Results are summarized in Table 3

TABLE 3

| Marker | Sample | Approximate Sens/Spec (%) | AUC |
|---|---|---|---|
| GSTP | WU | 38/93 | .66 |
| RARβ | WU | 31/93 | .58 |
| APC | WU | 35/98 | .64 |
| GSTP | Sediment | 43/90 | .64 |
| RARβ | Sediment | 56/77 | .64 |
| APC | Sediment | 52/87 | .62 |

GSTP by itself gave the best results by ROC analysis when samples were whole urine (neat). Spinning the sample down to sediments improved the performance of the RARβ Marker tremendously as shown by this same analysis.

### Example 3

### Prostate Massage

Two additional samples sets were tested and analyzed to determine the effect of prostatic massage on the performance of the urine based assay. In the first sample set, 36 samples (20 known cancers) were obtained from patients having prostatic massage limited to less than 20 seconds. In the other sample set, 77 samples (30 known cancers) were obtained from patients having prostatic massage for more than 20 seconds. In each case, samples were stored for five days or less. The results of the MSPCR conducted on these samples are summarized in Table 4.

TABLE 4

| | | | Massage | Sensitivity | Specificity |
|---|---|---|---|---|---|
| GSTP | RARβ2 | APC | (seconds) | (%) | (%) |
| X | | | <20 | 39 | 100 |
| | | X | <20 | 33 | 93 |
| | | X | <20 | 39 | 80 |
| | X | | <20 | 50 | 87 |
| X | | X | <20 | 56 | 93 |
| X | X | | <20 | 56 | 87 |
| X | X | X | <20 | 61 | 87 |
| X | X | X | <20 | 61 | 93 |
| X | | | >20 | 33 | 93 |
| | | X | >20 | 37 | 91 |
| | | X | >20 | 41 | 76 |
| | X | | >20 | 37 | 84 |
| X | | X | >20 | 48 | 89 |
| X | X | | >20 | 41 | 82 |
| X | X | X | >20 | 48 | 82 |
| X | X | X | >20 | 52 | 82 |

The best results were obtained from the panel that included GSTP, RARβ, and APC when the prostate massage was less than 20 seconds in duration. This is surprising as one would have expected lengthier massage to release more cells and increase, at the least, the specificity.

### Example 4

### Digital Rectal Examination

Four additional sample sets were tested and analyzed to determine the effect of selection on the basis of an abnormal versus a normal digital rectal examination (DRE) on the performance of the urine based assay. In the first sample set, 64 whole urine samples (23 known cancers) were obtained from patients having a normal DRE. In the second sample set 33 whole urine samples (19 known cancers) were obtained from patients having an abnormal DRE. The third sample set contained 48 sedimented samples (21 known cancers) who presented with a normal DRE and the fourth sample set contained 22 sedimented samples (8 known cancers) of from patients with abnormal DREs. In each case, samples were stored for five days or less. The results of the MSPCR conducted on these samples are summarized in Table 5.

TABLE 5

| GSTP | RARβ2 | APC | Sample | DRE | Sens (%) | Spec (%) |
|---|---|---|---|---|---|---|
| X | | | Whole | Negative | 20 | 95 |
| | | X | Whole | Negative | 30 | 87 |
| | X | | Whole | Negative | 25 | 79 |
| X | | X | Whole | Negative | 35 | 85 |
| X | X | | Whole | Negative | 25 | 82 |
| X | X | X | Whole | Negative | 40 | 72 |
| X | X | X | Whole | Negative | 40 | 74 |
| X | | | Whole | Abnormal | 60 | 92 |
| | | X | Whole | Abnormal | 60 | 92 |
| | X | | Whole | Abnormal | 53 | 85 |
| X | | X | Whole | Abnormal | 80 | 92 |
| X | X | | Whole | Abnormal | 67 | 85 |
| X | | | Sediment | Negative | 19 | 88 |
| | X | | Sediment | Negative | 10 | 88 |
| | X | | Sediment | Negative | 48 | 80 |
| | | X | Sediment | Negative | 33 | 76 |
| X | X | | Sediment | Negative | 52 | 76 |
| X | | X | Sediment | Negative | 38 | 72 |
| X | | | Sediment | Abnormal | 71 | 85 |
| X | | | Sediment | Abnormal | 86 | 77 |
| | X | | Sediment | Abnormal | 57 | 62 |
| | X | | Sediment | Abnormal | 57 | 85 |
| | | X | Sediment | Abnormal | 86 | 92 |
| X | X | | Sediment | Abnormal | 71 | 77 |
| X | | X | Sediment | Abnormal | 86 | 75 |

Markers used with samples selected from patients with an abnormal DRE performed substantially better than the cases in which patients had negative DREs. Whole urine samples from patients with abnormal DREs were assayed with nearly the same degree of sensitivity and specificity as the best sedimented samples when the Marker panel was made up of GSTP and APC. A single Marker (APC) assay performed the best in sedimented samples with an abnormal DRE but the GSTP/APC panel was not far behind.

### Example 5

### PSA Level

Two additional samples sets were tested and analyzed to determine the effect of sample selection based on PSA level. In the first sample set, −52 whole urine samples (25 known cancers) were obtained from patients having a PSA value of 2.5-4 ng/ml. In the other sample set, 169 samples (80 known cancers) were obtained from patients having a PSA level of 4-10 ng/ml. In each case, samples were stored within five days at 4° C. between urine collection and sedimentation procedures. The results of the MSPCR conducted on these samples is summarized in Table 6 below.

For 52 subjects with PSA levels between 2.5 and 4 ng/mL (including 25 cancer and 27 non-cancer cases), sensitivity of 58% and specificity of 88% was demonstrated when using logistic regression model, or 58% and 81%, respectively, when using 3 markers with the following Ct cutoffs: GSTP=26, RAR=28, APC=25 and no test rate at 3.8%. For patient co-hort with PSA 4-10, sensitivity/specificity characteristics were 59/65% using the same markers and Ct cutoffs. Two-sample T-test confirmed that there was no statistically significant difference in assay performance between two co-horts with PSA ranges 2.5-4 and 4-10 ng/mL (P=0.000). Results are shown in Table 6.

TABLE 6

| | | | | | | | No test |
|---|---|---|---|---|---|---|---|
| Marker | GST D2 | RAR | APC | Sens, % | Spec, % | AUC | rate, % |
| PSA 2.5-4, | 28 | | | 38 | 85 | 0.619 | 3.8 |
| n = 52, | 26 | | | 26 | 96 | | |
| C = 25, | | 28 | | 54 | 85 | 0.712 | |
| NC = 27 | | | 26 | 25 | 81 | 0.574 | |
| | 26 | | 28 | 54 | 73 | 0.622 | |
| | Logistic | | | 38 | 85 | | |
| | regression model | | | | | | |
| | 26 | 28 | | 54 | 81 | 0.697 | |
| | Logistic | | | 58 | 85 | | |
| | regression model | | | | | | |
| | 26 | 27 | 25 | 50 | 81 | 0.688 | |
| | 26 | 28 | 25 | 58 | 81 | | |
| | Logistic | | | 58 | 88 | | |
| | regression model | | | | | | |
| PSA 4-10, | 28 | | | 49 | 90 | 0.652 | 5.9 |
| n = 169, | 26/30 | | | 34/44 | 95/84 | | |
| C = 80, | | 28/26 | | 48/40 | 70/84 | 0.602 | |
| NC = 89 | | | 26 | 40 | 83 | 0.616 | |
| | 26 | | 28 | 62 | 70 | 0.634 | |
| | 28 | | 27 | 59 | 74 | | |
| | Logistic | | | 45 | 85 | | |
| | regression model | | | | | | |
| | 26 | 28 | | 53 | 70 | 0.644 | |
| | Logistic | | | 45 | 85 | | |
| | regression model | | | | | | |
| | 26 | 28 | 25 | 59 | 65 | 0.651 | |
| | 26 | 27 | 25 | 56 | 73 | | |

| | | |
|---|---|---|
| Logistic | 45 | 87 |
| regression model | | |

### Example 6

### More Extensive Multiplexing

More extensive multiplexing was conducted using additional markers known to be useful in prostate cancer analysis. Maximum assay specificity was sought given that the objective of the assay was to resolve ambiguous PSA results (2.5-4 ng/ml in clinical use). The assays were performed on sediment samples stored for 3 days. One round of PCR was conducted on each of 60 samples (30 from cancer cases, 30 from non-cancer samples). Data with urine sediments generated for 6 markers

(GST+RAR+APC+RASS+CDH1+PDLIM4) using 3 quadruplex reactions demonstrate that CDH1, RASS and PDLIM4 do not add value to maintain specificity at 80%. Inclusion of all 6 markers impairs assay specificity (sens=44%, spec=63%). Moreover, use of 6 markers does not suit the singletube assay format. Results are shown in Table 7.

TABLE 7

| GSTP1 | RAR | APC | RASSF1A | CDH1D2 | PDLM4D2 | Sens | Spec |
|---|---|---|---|---|---|---|---|
| X | X | | X | | X | 41% | 80% |
| X | X | | | X | X | 44% | 70% |
| X | X | | X | | X | 37% | 83% |
| X | X | | | | X | 41% | 73% |
| X | X | X | X | X | X | 44% | 63% |
| X | X | X | | | | 38% | 80% |

### Example 7

### Normalization of Results using S100A2

Urine samples were tested using four markers (GSTP1, RARβ2, APC, and S100A2) in MPCR reactions as described above.

TABLE 7

| | Pru-Mu | | |
|---|---|---|---|
| | GSTP1 | RAR | APC |
| Sensitivity | 20.00% | 5.00% | 5.00% |
| Specificity | 90.00% | 100.00% | 90.00% |

| | GSTP1/APC//RARB2 |
|---|---|
| Sensitivity | 20.00% |
| Specificity | 80.00% |

The data above show performance of three markers individually and as a combination on a representative set of 20 Cancers and 10 Non-cancers. The sensitivity is lower than typically observed due to the less than optimal storage time of the urine samples prior to sedimentation.

The same samples were then analyzed with S100 and the delta Ct between S100 and the gene of interest was used to generate a cutoff. The data show improved performance for RARβ2 and APC but not GSTP1. This differential effect was observed because the borderline cases for RARβ2 and APC could now be unambiguously assigned as Cancer, improving the sensitivity. There were no borderline cases for GSTP1 in this data set, however, which is why the sensitivity remained unchanged.

TABLE 8

| | Pru-Mu - Norm S100 | | |
|---|---|---|---|
| | GSTP1 | RAR | APC |
| Sensitivity | 20.00% | 15.00% | 20.00% |
| Specificity | 90.00% | 100.00% | 90.00% |

| | |
|---|---|
| | GSTP1/APC//RARB2 |
| Sensitivity | 40.00% |
| Specificity | 80.00% |

We claim:

1. A method of detecting prostate cancer comprising, obtaining a urine sample from a person, and determining the methylation status of only the GSTP1 gene and one or more controls in the urine sample no later than three days after said urine sample was obtained; wherein methylation that exceeds a pre-determined value is indicative of prostate cancer and methylation that does not exceed such pre-determined value is indicative of the absence of prostate cancer.

2. The method according to claim 1 further comprising measuring the presence of a reference Marker and wherein prior to the collection of said urine sample, the person is subjected to prostatic massage for about 20 seconds.

3. The method of claim 1 further comprising the steps of determining the PSA level of said person and conducting said method only on people having a PSA level between 2.5 and 4 ng/ml.

4. The method of claim 1 wherein the methylation status of the genes is determined using nested PCR wherein a first round of PCR is conducted followed by a subsequent round of PCR wherein primers and probes for conducting said subsequent PCR are directed to sequences within the sequences amplified in the first round of PCR and wherein the urine samples are spun down to form sediments prior to conducting the first round of PCR.

5. A method of detecting prostate cancer comprising, obtaining a urine sample from a person, determining the methylation status of only the S100 gene and only one more genes selected from the group consisting of the GSTP1 gene, APC, and RARβ2 and one or more controls in the urine sample no later than three days after said urine sample was obtained; wherein if the Ct value of the methylation of the GSTP1, APC, or RARβ2 gene less that of the S100 gene exceeds a pre-determined value it is indicative of prostate cancer and methylation that does not exceed such pre-determined value is indicative of the absence of prostate cancer.

6. The method according to claim 5 further comprising measuring the presence of a reference Marker and wherein prior to the collection of said urine sample, the person is subjected to prostatic massage for about 20 seconds.

7. The method of claim 5 further comprising the steps of determining the PSA level of said person and conducting said method only on people having a PSA level between 2.5 and 4 ng/ml.

8. The method of claim 5 wherein the methylation status of the genes is determined using nested PCR wherein a first round of PCR is conducted followed by a subsequent round of PCR wherein primers and probes for conducting said subsequent PCR are directed to sequences within the sequence amplified in the first round of PCR and wherein the urine samples are spun down to form sediments prior to conducting the first round of PCR.

9. A method of detecting prostate cancer comprising, obtaining a urine sample from a patient, determining the methylation status of only the GSTP1 gene, the RARβ1, the APC gene and one or more controls in the urine sample; wherein methylation that exceeds a pre-determined value is indicative of prostate cancer and methylation that does not exceed such pre-determined value is indicative of the absence of prostate cancer wherein said method is conducted in a single vessel that is left unopened during the conduct of the method.

10. The method according to claim 9 further comprising measuring the presence of a reference Marker.

11. The method of claim 9 further comprising the steps of determining the PSA level of said person and conducting said method only on people having a PSA level between 2.5 and 4 ng/ml.

12. The method of claim 9 wherein the methylation status of the genes is determined using nested PCR wherein a first round of PCR is conducted followed by a subsequent round of PCR wherein primers and probes for conducting said subsequent PCR are directed to sequences within the sequences amplified in the first round of PCR and wherein the urine samples are spun down to form sediments prior to conducting the first round of PCR.

13. The method of claim 9 wherein prior to the collection of said urine sample, the person is subjected to prostatic massage for about 20 seconds.

14. A method of detecting prostate cancer comprising, obtaining a urine sample from a patient with an abnormal DRE, determining the methylation status of only the GSTP1 gene, the RARPβ1, the APC gene and one or more controls in the urine sample; wherein methylation that exceeds a pre-determined value is indicative of prostate cancer and methylation that does not exceed such pre-determined value is indicative of the absence of prostate cancer wherein said method is conducted in a single vessel that is left unopened during the conduct of the method.

15. The method according to claim 14 further comprising measuring the presence of a reference Marker.

16. The method of claim 14 further comprising the steps of determining the PSA level of said person and conducting said method only on people having a PSA level between 2.5 and 4 ng/ml.

17. The method of claim 14 wherein the methylation status of the genes is determined using nested PCR wherein a first round of PCR is conducted followed by a subsequent round of PCR wherein primers and probes for conducting said subsequent PCR are directed to sequences within the sequences amplified in the first round of PCR and wherein the urine samples are spun down to form sediments prior to conducting the first round of PCR.

18. The method of claim 14 wherein prior to the collection of said urine sample, the person is subjected to prostatic massage for about 20 seconds.

19. A method of detecting prostate cancer comprising, obtaining a urine sample from a patient with an abnormal DRE, determining the methylation status of only the GSTP1 gene and the RARPβ1 and one or more controls in the urine sample; wherein methylation that exceeds a pre-determined value is indicative of prostate cancer and methylation that does not exceed such pre-determined value is indicative of the absence of prostate cancer wherein said method is conducted in a single vessel that is left unopened during the conduct of the method.

20. The method according to claim 19 further comprising measuring the presence of a reference Marker.

21. The method of claim 19 further comprising the steps of determining the PSA level of said person and conducting said method only on people having a PSA level between 2.5 and 4 ng/ml.

22. The method of claim 19 wherein the methylation status of the genes is determined using nested PCR wherein a first round of PCR is conducted followed by a subsequent round of PCR wherein primers and probes for conducting said subsequent PCR are directed to sequences within the sequences amplified in the first round of PCR and wherein the urine samples are spun down to form sediments prior to conducting the first round of PCR.

23. The method of claim 19 wherein prior to the collection of said urine sample, the person is subjected to prostatic massage for about 20 seconds.

24. A kit for conducting an assay to detect prostate cancer, comprising: nucleic acid amplification and detection reagents for detecting the presence of genes consisting essentially of GSTP1 and one or more control genes and instructions that direct its use in patients in having measured PSA levels of 2.5-4 ng/ml.

25. A kit for conducting an assay to detect prostate cancer, comprising: nucleic acid amplification and detection reagents for detecting the presence of genes consisting essentially of GSTP1, RARβ1 and one or more control genes and instructions that direct its use in patients in having measured PSA levels of 2.5-4 ng/ml.

26. A kit for conducting an assay to detect prostate cancer, comprising: nucleic acid amplification and detection reagents for detecting the presence of genes consisting essentially of GSTP, RARPβ1, APC and one or more control genes and instructions that direct its use in patients in having measured PSA levels of 2.5-4 ng/ml.

27. A kit for conducting an assay to detect prostate cancer, comprising: nucleic acid amplification and detection reagents for detecting the presence of the S100A2 gene and one more genes selected from the group consisting of GSTP, RARβ1, and APC, and one or more control genes.
